(19) United States
(12) Patent Application Publication
(10) Pub. No.: US 2021/0292272 A1
(43) Pub. Date: Sep. 23, 2021

(54) Salts of Zuclomiphene

(71) Applicant: Apotex Inc., Toronto (CA)

(72) Inventors: Fabio E. S. Souza, Brantford (CA); Fatemeh Mohammadpourmir, Brantford (CA); Alexander J. Stirk, Brantford (CA); Avedis Karadeolian, Brantford (CA); Allan W. Rey, Brantford (CA)

(21) Appl. No.: 17/206,362

(22) Filed: Mar. 19, 2021

Related U.S. Application Data
(60) Provisional application No. 62/992,535, filed on Mar. 20, 2020.

(51) Int. Cl. C07C 217/20

(52) U.S. Cl. CPC C07C 217/20

(57) ABSTRACT

The present invention provides salts of zuclomiphene and crystalline forms thereof. Specific salts of zuclomiphene provided by the present invention include sulphate, phosphate, succinate, L-tartrate, tosylate, L-malate, maleate, malonate, fumarate, glycolate, and hemi-citrate. Also provided are pharmaceutical compositions including the zuclomiphene salts and crystalline forms thereof and the use of these salts in the treatment of a disorder selected from the group including osteoporosis, bone fractures, loss of bone mineral density (BMD) and hot flashes in a subject suffering therefrom.

### CROSS-REFERENCE TO RELATED APPLICATION

This application claims the benefit of U.S. Provisional Patent Application No. 62/992,535, filed Mar. 20, 2020, the disclosure of which is hereby incorporated by reference in its entirety.

### BACKGROUND OF THE INVENTION

### Field of the Invention

The present invention is directed to novel salts of zuclomiphene and crystalline forms thereof, processes for the preparation thereof, pharmaceutical compositions containing these forms, and their use for the treatment of a disorder selected from the group consisting of osteoporosis, bone fractures, loss of bone mineral density (BMD) and hot flashes in a subject suffering therefrom.

### Description of Related Art

CLOMID®, a drug initially approved by the United States Food & Drug Administration in 1967 as an ovulatory stimulant, is an isomeric mixture of the citrate salts of cis-clomiphene (Z-clomiphene or ‘zuclomiphene’, (1-A)) and trans-clomiphene (E-clomiphene or ‘enclomiphene’, (1-B)) containing between 30% and 50% of the cis-isomer. Pure cis-isomer zuclomiphene, or (2-[4-[(Z)-2-chloro-1,2-diphenylethenyl]phenoxy]-N,N-diethylethanamine), in the form of the citrate salt, is currently under evaluation in clinical trials in the United States to treat hot flashes experienced by male patients with advanced prostate cancer undergoing androgen deprivation therapy (ADT).

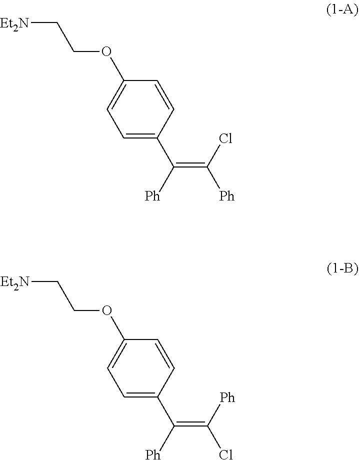

Salts of zuclomiphene, including the hydrochloride salt and the binaphthyl hydrogen phosphate (‘BPA’) salt, are reported in Palopoli et al. J. Med. Chem. 1967, 10 (1), 84-6, GB 1099093 A, and U.S. Pat. No. 3,848,030 A. FR 3082842 A1 refers to pharmaceutically acceptable salts of zuclomiphene derived from an inorganic acid or an organic acid. A list of possible zuclomiphene salts is provided which includes more than 130 salts, not including the reference to entire classes of salts such as carboxylates and sulfonates, which comprise innumerable members. Only vague directions as to the method of preparation of the salts are provided, such as by ‘conventional means’ and there is no specific guidance as to how to prepare and isolate any given salt, other than the citrate salt, or furthermore what the properties of any given salt may be.

The solubility of individual salt and crystalline forms of a drug substance in an aqueous environment often correlates with their relative bioavailability, since the manner in which the salt or crystalline form dissolves can correspond to the amount of the drug substance that is available to be absorbed into the body to provide the intended therapeutic effect. One measure of solubility is intrinsic dissolution rate (IDR), which is defined as the dissolution rate of a substance under constant surface area conditions. For low solubility substances, higher IDR values can correlate with higher bioavailability following administration. However, if the goal is to establish bioequivalence to an existing form of a drug under investigation, such as zuclomiphene citrate, substances with similar IDR values to the known form are preferred. Alternatively, for the development of extended or sustained release products, forms exhibiting lower IDR values are often preferable since they can provide slower dissolution of the drug independent of the excipients used in the formulation. Prediction of the solubility and IDR of an as yet undiscovered salt or crystalline form of a substance is currently not possible.

Different crystalline and/or salt forms of the same compound may have different crystal packing, thermodynamic, spectroscopic, kinetic, surface and mechanical properties. For example, different salts and/or crystalline forms may have different stability properties such that a particular crystalline form may be less sensitive to heat, relative humidity (RH) and/or light. Different salts and/or crystalline forms of a compound may also be more susceptible to moisture uptake, resulting in a potential alteration of physical characteristics of the form such as flowability, density or compressibility, which can lead to problems during formulation/tabletting and/or to changes in dissolution rate of the formulated drug product.

For example, a particular salt and/or crystalline form may provide more favourable compressibility and/or density properties, thereby providing more desirable characteristics for formulation and/or product manufacturing. Differences in stability between salts and/or crystalline forms of a drug may result from changes in chemical reactivity, such as differential oxidation. Such properties may provide for more suitable product qualities, including a dosage form that is more resistant to discolouration when comprised of a specific salt and/or crystalline form. Particular salts and/or crystalline forms may also have different solubilities, thereby providing different pharmacokinetic parameters, which allow for specific salts and/or crystalline forms to be used in order to achieve specific pharmacokinetic targets.

Although general approaches to salt and crystalline form screening of active pharmaceutical ingredients are known, it is well established that the prediction of whether any given compound will exhibit polymorphism is not possible. Accordingly, it is not possible to extend generalities to the number and kinds of crystalline forms that can exist for zuclomiphene salts, or to what methods will be suitable for the preparation of any given form. Furthermore, prediction of the properties of any unknown salts and/or crystalline forms, and how they will differ from other crystalline forms or salts of the same compound, remains elusive (Joel Bernstein, Polymorphism in Molecular Crystals, Oxford University Press, New York, 2002, page 9).

There exists a need for novel salts of zuclomiphene and crystalline forms thereof for use in improved drug products containing zuclomiphene and their manufacture.

### SUMMARY OF THE INVENTION

The zuclomiphene salts and crystalline forms of the present invention comprise pharmaceutically acceptable acids. Further, embodiments of the present invention incorporate first class acids according to a notable reference book on the pharmaceutical acceptability of salts: P. Heinrich Stahl, Camille G. Wermuth (Eds.), Handbook of Pharmaceutical Salts Properties, Selection, and Use; 2002. First class acids are classified by Stahl as those that afford physiologically ubiquitous ions or metabolites in biochemical pathways, supporting their unrestricted use in pharmaceuticals.

The zuclomiphene salts and crystalline forms of the present invention exhibit differences in properties when compared to the known salts and crystalline forms of zuclomiphene, such as the citrate salt in the drug product currently under evaluation in clinical trials in the United States. Depending on the specific salts and crystalline forms of the invention used, properties that may differ between the invention and known salt and crystalline forms of zuclomiphene include the following: packing properties such as molar volume, density and hygroscopicity, thermodynamic properties such as melting point and solubility, kinetic properties such as intrinsic dissolution rate and chemical/crystalline form stability, surface properties such as crystal habit and mechanical properties such as hardness, tensile strength, compactibility, tableting, handling, flow, and blending. Furthermore, the salts and crystalline forms of the present invention may be prepared by facile and industrially advantageous processes.

Depending on the specific salt and crystalline form of the invention used, the present invention provides opportunities for achieving specific formulation and dosage form goals. For example, salt and crystalline forms are provided that exhibit higher solubility compared to zuclomiphene citrate salt, which can be used to provide products with enhanced bioavailability. Salt and crystalline forms are also provided having comparable solubility to this form, which are preferable when trying to achieve bioequivalence between dosage forms.

Thus, the present invention provides new salt and crystalline forms of zuclomiphene having advantages over known forms of zuclomiphene that can be exploited in the development of new formulations and dosage forms containing zuclomiphene.

Embodiments of the present invention are free-flowing, crystalline solids, which provides advantages in handling and formulation. Additionally, embodiments of the present invention exhibit stability when exposed to conditions of 40° C./75% RH.

Accordingly, in a first aspect of the present invention, there is provided a sulphate salt of zuclomiphene. In a preferred embodiment of the first aspect, the molar ratio of zuclomiphene to sulphuric acid is approximately 1:1. In a more preferred embodiment of the first aspect, the salt is characterized by a PXRD diffractogram comprising peaks, expressed in degrees 2θ (±0.2°), at 5.2°, 14.6° and 20.6°. More preferably, the salt of the first aspect is characterized by a PXRD diffractogram further comprising at least three peaks, expressed in degrees 2θ (±0.2°), selected from the group consisting of: 10.0°, 10.3°, 12.2°, 12.4°, 12.9°, 15.1°, 16.0°, 17.4°, 22.2° and 22.9°. In a further preferred embodiment of the first aspect, the PXRD diffractogram further comprises peaks, expressed in degrees 2θ (±0.2°), at 10.0°, 10.3°, 12.2°, 12.4°, 12.9°, 15.1°, 16.0°, 17.4°, 22.2° and 22.9°. Preferably, the salt of the first aspect of the invention provides a PXRD diffractogram comprising peaks in substantially the same positions (±0.2° 2θ) as those shown in FIG. 1. In a further preferred embodiment of the first aspect, the salt is characterized by a DSC thermogram comprising an endothermic peak with a peak onset at approximately 152° C. and a peak maximum at approximately 155° C. Preferably, the salt of the first aspect is characterized by a DSC thermogram that is substantially the same in appearance as the DSC thermogram provided in FIG. 12.

In a second aspect of the present invention, there is provided a phosphate salt of zuclomiphene. In a preferred embodiment of the second aspect, the molar ratio of zuclomiphene to phosphoric acid is approximately 1:1. In a more preferred embodiment of the second aspect, the salt is characterized by a PXRD diffractogram comprising peaks, expressed in degrees 2θ (±0.2°), at 4.5°, 9.0° and 19.3°. More preferably, the salt of the second aspect is characterized by a PXRD diffractogram further comprising at least three peaks, expressed in degrees 2θ (±0.2°), selected from the group consisting of: 11.0°, 12.5°, 13.5°, 13.8°, 15.0°, 17.7°, 19.3°, 20.6°, 22.3° and 24.2°. In a further preferred embodiment of the second aspect, the PXRD diffractogram further comprises peaks, expressed in degrees 2θ (±0.2°), at 11.0°, 12.5°, 13.5°, 13.8°, 15.0°, 17.7°, 19.3°, 20.6°, 22.3° and 24.2°. Preferably, the salt of the second aspect of the invention provides a PXRD diffractogram comprising peaks in substantially the same positions (±0.2° 2θ) as those shown in FIG. 2. In a further preferred embodiment of the second aspect, the salt is characterized by a DSC thermogram comprising an endothermic peak with a peak onset at approximately 131° C. and a peak maximum at approximately 133° C. Preferably, the salt of the second aspect is characterized by a DSC thermogram that is substantially the same in appearance as the DSC thermogram provided in FIG. 13.

In a third aspect of the present invention, there is provided a succinate salt of zuclomiphene. In a preferred embodiment of the third aspect, the molar ratio of zuclomiphene to succinic acid is approximately 1:1. In a more preferred embodiment of the third aspect, the salt is characterized by a PXRD diffractogram comprising peaks, expressed in degrees 2θ (±0.2°), at 5.5°, 10.1° and 17.3°. More preferably, the salt of the third aspect is characterized by a PXRD diffractogram further comprising at least three peaks, expressed in degrees 2θ (±0.2°), selected from the group consisting of: 11.8°, 13.9°, 15.2°, 18.6°, 19.7°, 20.1°, 21.4°, 22.1°, 22.9° and 23.7°. In a further preferred embodiment of the third aspect, the PXRD diffractogram further comprises peaks, expressed in degrees 2θ (±0.2°), at 11.8°, 13.9°, 15.2°, 18.6°, 19.7°, 20.1°, 21.4°, 22.1°, 22.9° and 23.7°. Preferably, the salt of the third aspect of the invention provides a PXRD diffractogram comprising peaks in substantially the same positions (±0.2° 2θ) as those shown in FIG. 3. In a further preferred embodiment of the third aspect, the salt is characterized by a DSC thermogram comprising an endothermic peak with a peak onset at approximately 101° C. and a peak maximum at approximately 104° C. Preferably, the salt of the third aspect is characterized by a DSC thermogram that is substantially the same in appearance as the DSC thermogram provided in FIG. 14.

In a fourth aspect of the present invention, there is provided an L-tartrate salt of zuclomiphene. In a preferred embodiment of the fourth aspect, the molar ratio of zuclomiphene to L-tartaric acid is approximately 1:1. In a more preferred embodiment of the fourth aspect, the salt is characterized by a PXRD diffractogram comprising peaks, expressed in degrees 2θ (±0.2°), at 6.0°, 9.0° and 12.0°. More preferably, the salt of the fourth aspect is characterized by a PXRD diffractogram further comprising at least three peaks, expressed in degrees 2θ (±0.2°), selected from the group consisting of: 11.4°, 13.7°, 14.9°, 15.9°, 17.4°, 18.3°, 18.9°, 20.4°, 20.9° and 22.4°. In a further preferred embodiment of the fourth aspect, the PXRD diffractogram further comprises peaks, expressed in degrees 2 θ (±0.2°), at 11.4°, 13.7°, 14.9°, 15.9°, 17.4°, 18.3°, 18.9°, 20.4°, 20.9° and 22.4°. Preferably, the salt of the fourth aspect of the invention provides a PXRD diffractogram comprising peaks in substantially the same positions (±0.2° 2θ) as those shown in FIG. 4. In a further preferred embodiment of the fourth aspect, the salt is characterized by a DSC thermogram comprising an endothermic peak with a peak onset at approximately 132° C. and a peak maximum at approximately 135° C. Preferably, the salt of the fourth aspect is characterized by a DSC thermogram that is substantially the same in appearance as the DSC thermogram provided in FIG. 15.

In a fifth aspect of the present invention, there is provided a tosylate salt of zuclomiphene. In a preferred embodiment of the fifth aspect, the molar ratio of zuclomiphene to p-toluenesulfonic acid is approximately 1:1. In a more preferred embodiment of the fifth aspect, the salt is characterized by a PXRD diffractogram comprising peaks, expressed in degrees 2 θ (±0.2°), at 5.6°, 11.1° and 18.1°. More preferably, the salt of the fifth aspect is characterized by a PXRD diffractogram further comprising at least three peaks, expressed in degrees 2 θ (±0.2°), selected from the group consisting of: 5.0°, 8.6°, 10.4°, 13.3°, 14.0°, 16.8°, 19.3°, 21.9°, 22.8° and 24.6°. In a further preferred embodiment of the fifth aspect, the PXRD diffractogram further comprises peaks, expressed in degrees 2 θ (±0.2°), at 5.0°, 8.6°, 10.4°, 13.3°, 14.0°, 16.8°, 19.3°, 21.9°, 22.8° and 24.6°. Preferably, the salt of the fifth aspect of the invention provides a PXRD diffractogram comprising peaks in substantially the same positions (±0.2° 2θ) as those shown in FIG. 5. In a further preferred embodiment of the fifth aspect, the salt is characterized by a DSC thermogram comprising an endothermic peak with a peak onset at approximately 163° C. and a peak maximum at approximately 164° C. Preferably, the salt of the fifth aspect is characterized by a DSC thermogram that is substantially the same in appearance as the DSC thermogram provided in FIG. 16.

In a sixth aspect of the present invention, there is provided an L-malate salt of zuclomiphene. In a preferred embodiment of the sixth aspect, the molar ratio of zuclomiphene to L-malic acid is approximately 1:1. In a more preferred embodiment of the sixth aspect, the salt is characterized by a PXRD diffractogram comprising peaks, expressed in degrees 2θ (±0.2°), at 6.4°, 12.8° and 22.5°. More preferably, the salt of the sixth aspect is characterized by a PXRD diffractogram further comprising at least three peaks, expressed in degrees 2θ (±0.2°), selected from the group consisting of: 9.6°, 10.1°, 12.0°, 14.0°, 15.6°, 16.0°, 16.6°, 18.1°, 18.7° and 19.0°. In a further preferred embodiment of the sixth aspect, the PXRD diffractogram further comprises peaks, expressed in degrees 2θ (±0.2°), at 9.6°, 10.1°, 12.0°, 14.0°, 15.6°, 16.0°, 16.6°, 18.1°, 18.7° and 19.0°. Preferably, the salt of the sixth aspect of the invention provides a PXRD diffractogram comprising peaks in substantially the same positions (±0.2° 2θ) as those shown in FIG. 6. In a further preferred embodiment of the sixth aspect, the salt is characterized by a DSC thermogram comprising an endothermic peak with a peak onset at approximately 112° C. and a peak maximum at approximately 116° C. Preferably, the salt of the sixth aspect is characterized by a DSC thermogram that is substantially the same in appearance as the DSC thermogram provided in FIG. 17.

In a seventh aspect of the present invention, there is provided a maleate salt of zuclomiphene. In a preferred embodiment of the seventh aspect, the molar ratio of zuclomiphene to maleic acid is approximately 1:1. In a more preferred embodiment of the seventh aspect, the salt is characterized by a PXRD diffractogram comprising peaks, expressed in degrees 2θ(±0.2°), at 6.6°, 13.2° and 20.5°. More preferably, the salt of the seventh aspect is characterized by a PXRD diffractogram further comprising at least three peaks, expressed in degrees 2 θ (±0.2°), selected from the group consisting of: 12.1°, 14.2°, 15.0°, 16.1°, 16.5°, 17.8°, 18.2°, 19.5°, 19.8° and 22.3°. In a further preferred embodiment of the seventh aspect, the PXRD diffractogram further comprises peaks, expressed in degrees 2θ (±0.2°), at 12.1°, 14.2°, 15.0°, 16.1°, 16.5°, 17.8°, 18.2°, 19.5°, 19.8° and 22.3°. Preferably, the salt of the seventh aspect of the invention provides a PXRD diffractogram comprising peaks in substantially the same positions (±0.2° 2θ) as those shown in FIG. 7. In a further preferred embodiment of the seventh aspect, the salt is characterized by a DSC thermogram comprising an endothermic peak with a peak onset at approximately 119° C. and a peak maximum at approximately 121° C. Preferably, the salt of the seventh aspect is characterized by a DSC thermogram that is substantially the same in appearance as the DSC thermogram provided in FIG. 18.

In an eighth aspect of the present invention, there is provided a malonate salt of zuclomiphene. In a preferred embodiment of the eighth aspect, the molar ratio of zuclomiphene to malonic acid is approximately 1:1. In a more preferred embodiment of the eighth aspect, the salt is characterized by a PXRD diffractogram comprising peaks, expressed in degrees 2θ (±0.2°), at 6.8°, 13.6° and 18.2°. More preferably, the salt of the eighth aspect is characterized by a PXRD diffractogram further comprising at least three peaks, expressed in degrees 2θ (±0.2°), selected from the group consisting of: 11.3°, 15.6°, 17.0°, 19.9°, 20.4°, 21.4°, 22.0°, 22.9°, 23.7° and 26.1°. In a further preferred embodiment of the eighth aspect, the PXRD diffractogram further comprises peaks, expressed in degrees 2θ (±0.2°), at 11.3°, 15.6°, 17.0°, 19.9°, 20.4°, 21.4°, 22.0°, 22.9°, 23.7° and 26.1°. Preferably, the salt of the eighth aspect of the invention provides a PXRD diffractogram comprising peaks in substantially the same positions (±0.2° 2θ) as those shown in FIG. 8. In a further preferred embodiment of the eighth aspect, the salt is characterized by a DSC thermogram comprising an endothermic peak with a peak onset at approximately 114° C. and a peak maximum at approximately 116° C. Preferably, the salt of the eighth aspect is characterized by a DSC thermogram that is substantially the same in appearance as the DSC thermogram provided in FIG. 19.

In a ninth aspect of the present invention, there is provided a fumarate salt of zuclomiphene. In a preferred embodiment of the ninth aspect, the molar ratio of zuclomiphene to fumaric acid is approximately 1:1. In a more preferred embodiment of the ninth aspect, the salt is characterized by a PXRD diffractogram comprising peaks, expressed in degrees 2θ (±0.2°), at 6.9°, 13.9° and 17.9°. More preferably, the salt of the ninth aspect is characterized by a PXRD diffractogram further comprising at least three peaks, expressed in degrees 2θ (±0.2°), selected from the group consisting of: 10.0°, 10.5°, 15.6°, 16.3°, 17.4°, 19.3° and 21.1°. In a further preferred embodiment of the ninth aspect, the PXRD diffractogram further comprises peaks, expressed in degrees 2θ (±0.2°), at 10.0°, 10.5°, 15.6°, 16.3°, 17.4°, 19.3° and 21.1°. Preferably, the salt of the ninth aspect of the invention provides a PXRD diffractogram comprising peaks in substantially the same positions (±0.2° 2θ) as those shown in FIG. 9. In a further preferred embodiment of the ninth aspect, the salt is characterized by a DSC thermogram comprising an endothermic peak with a peak onset at approximately 110° C. and a peak maximum at approximately 113° C. Preferably, the salt of the ninth aspect is characterized by a DSC thermogram that is substantially the same in appearance as the DSC thermogram provided in FIG. 20.

In a tenth aspect of the present invention, there is provided a glycolate salt of zuclomiphene. In a preferred embodiment of the tenth aspect, the molar ratio of zuclomiphene to glycolic acid is approximately 1:1. In a more preferred embodiment of the tenth aspect, the salt is characterized by a PXRD diffractogram comprising peaks, expressed in degrees 2θ (±0.2)°, at 6.0°, 9.0° and 18.2°. More preferably, the salt of the tenth aspect is characterized by a PXRD diffractogram further comprising at least three peaks, expressed in degrees 2θ (±0.2°), selected from the group consisting of: 9.7°, 10.5°, 12.0°, 15.2°, 15.9°, 17.3°, 18.2°, 20.0°, 21.2° and 23.5°. In a further preferred embodiment of the tenth aspect, the PXRD diffractogram further comprises peaks, expressed in degrees 2θ (±0.2)°, at 9.7°, 10.5°, 12.0°, 15.2°, 15.9°, 17.3°, 18.2°, 20.0°, 21.2° and 23.5°. Preferably, the salt of the tenth aspect of the invention provides a PXRD diffractogram comprising peaks in substantially the same positions (±0.2° 2θ) as those shown in FIG. 10. In a further preferred embodiment of the tenth aspect, the salt is characterized by a DSC thermogram comprising an endothermic peak with a peak onset at approximately 63° C. and a peak maximum at approximately 68° C. Preferably, the salt of the tenth aspect is characterized by a DSC thermogram that is substantially the same in appearance as the DSC thermogram provided in FIG. 21.

In an eleventh aspect of the present invention, there is provided a hemi-citrate salt of zuclomiphene. In a preferred embodiment of the eleventh aspect, the molar ratio of zuclomiphene to citric acid is approximately 1:0.5. In a more preferred embodiment of the eleventh aspect, the salt is characterized by a PXRD diffractogram comprising peaks, expressed in degrees 2θ (±0.2°), at 5.0°, 13.3° and 16.8°. More preferably, the salt of the eleventh aspect is characterized by a PXRD diffractogram further comprising at least three peaks, expressed in degrees 2θ (±0.2°), selected from the group consisting of: 9.5°, 10.9°, 14.6°, 15.7°, 18.2°, 20.2°, 20.9°, 21.6° and 24.0°. In a further preferred embodiment of the eleventh aspect, the PXRD diffractogram further comprises peaks, expressed in degrees 2θ (±0.2°), at 9.5°, 10.9°, 14.6°, 15.7°, 18.2°, 20.2°, 20.9°, 21.6° and 24.0°. Preferably, the salt of the eleventh aspect of the invention provides a PXRD diffractogram comprising peaks in substantially the same positions (±0.2° 2θ) as those shown in FIG. 11. In a further preferred embodiment of the eleventh aspect, the salt is characterized by a DSC thermogram comprising an endothermic peak with a peak onset at approximately 82° C. and a peak maximum at approximately 86° C. Preferably, the salt of the eleventh aspect is characterized by a DSC thermogram that is substantially the same in appearance as the DSC thermogram provided in FIG. 22.

In a twelfth aspect of the present invention, there is provided a pharmaceutical composition comprising a salt of zuclomiphene according to the first, second, third, fourth, fifth, sixth, seventh, eighth, ninth, tenth, or eleventh aspects of the invention, and one or more pharmaceutically acceptable excipients. Preferably, the pharmaceutical composition is in the form of a solid oral dosage form. Most preferably, the pharmaceutical composition is a capsule or a tablet. Preferably, the pharmaceutical composition of the twelfth aspect comprises an amount of the zuclomiphene salt of the first, second, third, fourth, fifth, sixth, seventh, eighth, ninth, tenth, or eleventh aspects that is equivalent to 50 mg zuclomiphene citrate.

In a thirteenth aspect of the present invention, there is provided the use of a salt of zuclomiphene according to the first, second, third, fourth, fifth, sixth, seventh, eighth, ninth, tenth, or eleventh aspects of the invention, or the pharmaceutical compositions of the twelfth aspect of the invention, in the treatment of a disorder selected from the group consisting of osteoporosis, bone fractures, loss of bone mineral density (BMD) and hot flashes. In a preferred embodiment of the thirteenth aspect, the disorder is hot flashes. In a further preferred embodiment of the thirteenth aspect, the treatment comprises suppressing or inhibiting hot flashes in a male patient undergoing androgen deprivation therapy for the treatment of prostate cancer.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention are described, by way of example only, with reference to the attached Figures.

FIG. 1 is a representative PXRD diffractogram of zuclomiphene sulphate Form APO-I as prepared in Example 1.

FIG. 2 is a representative PXRD diffractogram of zuclomiphene phosphate Form APO-I as prepared in Example 2.

FIG. 3 is a representative PXRD diffractogram of zuclomiphene succinate Form APO-I as prepared in Example 3.

FIG. 4 is a representative PXRD diffractogram of zuclomiphene L-tartrate Form APO-I as prepared in Example 4.

FIG. 5 is a representative PXRD diffractogram of zuclomiphene tosylate Form APO-I as prepared in Example 5.

FIG. 6 is a representative PXRD diffractogram of zuclomiphene L-malate Form APO-I as prepared in Example 6.

FIG. 7 is a representative PXRD diffractogram of zuclomiphene maleate Form APO-I as prepared in Example 7.

FIG. 8 is a representative PXRD diffractogram of zuclomiphene malonate Form APO-I as prepared in Example 8.

FIG. 9 is a representative PXRD diffractogram of zuclomiphene fumarate Form APO-I as prepared in Example 9.

FIG. 10 is a representative PXRD diffractogram of zuclomiphene glycolate Form APO-I as prepared in Example 10.

FIG. 11 is a representative PXRD diffractogram of zuclomiphene hemi-citrate Form APO-I as prepared in Example 11.

FIG. 12 is a representative DSC thermogram of zuclomiphene sulphate Form PO-I as prepared in Example 1.

FIG. 13 is a representative DSC thermogram of zuclomiphene phosphate Form APO-I as prepared in Example 2.

FIG. 14 is a representative DSC thermogram of zuclomiphene succinate Form APO-I as prepared in Example 3.

FIG. 15 is a representative DSC thermogram of zuclomiphene L-tartrate Form APO-I as prepared in Example 4.

FIG. 16 is a representative DSC thermogram of zuclomiphene tosylate Form APO-I as prepared in Example 5.

FIG. 17 is a representative DSC thermogram of zuclomiphene L-malate Form APO-I as prepared in Example 6.

FIG. 18 is a representative DSC thermogram of zuclomiphene maleate Form APO-I as prepared in Example 7.

FIG. 19 is a representative DSC thermogram of zuclomiphene malonate Form APO-I as prepared in Example 8.

FIG. 20 is a representative DSC thermogram of zuclomiphene fumarate Form APO-I as prepared in Example 9.

FIG. 21 is a representative DSC thermogram of zuclomiphene glycolate Form APO-I as prepared in Example 10.

FIG. 22 is a representative DSC thermogram of zuclomiphene hemi-citrate Form APO-I as prepared in Example 11.

### DETAILED DESCRIPTION

The present invention provides novel salts of zuclomiphene and crystalline forms thereof providing improved properties over known salts of zuclomiphene.

The zuclomiphene salts and crystalline forms of the present invention exhibit differences in properties when compared to known salts of zuclomiphene. Depending on the specific salts and crystalline forms of the invention used, properties that differ between the invention and known salts of zuclomiphene include crystal packing properties such as molar volume, density and hygroscopicity; thermodynamic properties such as melting point and solubility; kinetic properties such as dissolution rate and chemical/polymorphic stability; surface properties such as crystal habit/particle morphology; and/or mechanical properties such as hardness, tensile strength, compactibility, tabletting, handling, flow, and blending. The improved properties provided by the salts and crystalline forms of the present invention provide practical advantages over known forms of zuclomiphene that can be exploited to meet specific needs in the manufacture and formulation of zuclomiphene.

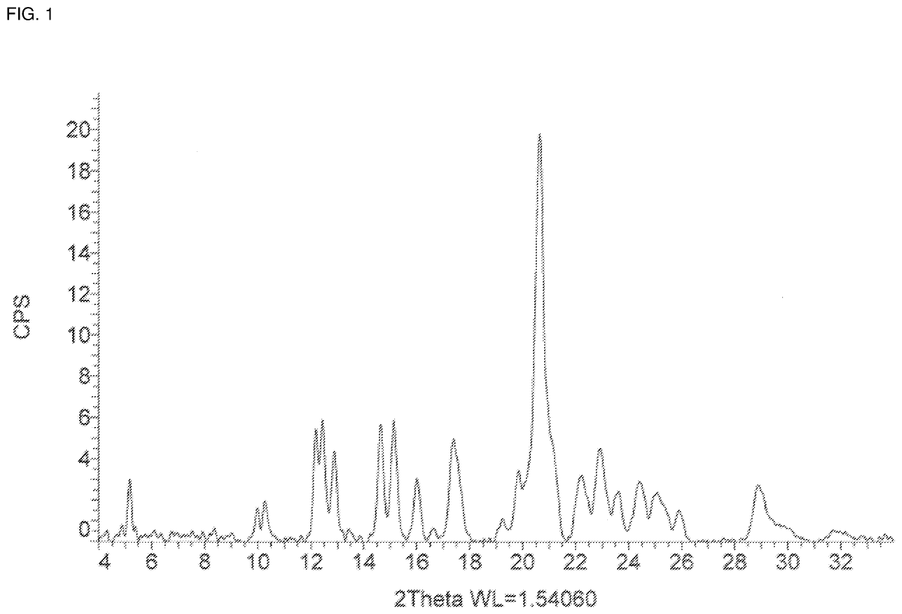

Depending on the manner in which the crystalline forms of the present invention are prepared, and the methodology and instrument used for PXRD analysis, the intensity of a given peak observed in a PXRD diffractogram of a crystalline form may vary when compared to the same peak in the representative PXRD diffractograms provided in FIGS. 1 to 11. Thus, differences in relative peak intensities between peaks in a PXRD diffractogram for a given crystalline form may be observed when compared to the relative peak intensities of the peaks in the representative PXRD diffractograms of FIGS. 1 to 11. Any such differences may be due, in part, to the preferred orientation of the sample and its deviation from the ideal random sample orientation, the preparation of the sample for analysis, and the methodology applied for the analysis. Such variations are known and understood by a person of skill in the art, and any such variations do not depart from the invention disclosed herein.

In addition to the differences in relative peak intensities that may be observed in comparison to the representative PXRD diffractograms provided in FIGS. 1 to 11, it is understood that individual peak positions may vary between ±0.2° 2θ from the values observed in the representative PXRD diffractograms provided in FIGS. 1 to 11 for the crystalline form of the invention, or listed in Tables 1 to 11. Such variations are known and understood by a person of skill in the art, and any such variations do not depart from the invention disclosed herein.

Further, depending on the instrument used for X-ray analysis and its calibration, uniform offsets in the peak position of each peak in a PXRD diffractogram of greater that 0.2° 2θ may be observed when compared to the representative PXRD diffractograms provided in FIGS. 1 to 11. Thus, PXRD diffractograms of the crystalline form of the present invention may, in some circumstances, display the same relative peak positions as observed in the representative PXRD diffractograms provided in FIGS. 1 to 11, with the exception that each peak is offset in the same direction, and by approximately the same amount, such that the overall PXRD diffractogram is substantially the same in appearance as the PXRD diffractograms of FIGS. 1 to 11, with the exception of the uniform offset in peak positions. The observation of any such uniform peak shift in a PXRD diffractogram does not depart from the invention disclosed herein given that the relative peak positions of the individual peaks within the PXRD diffractogram remain consistent with the relative peak positions observed in the PXRD diffractograms of FIGS. 1 to 11.

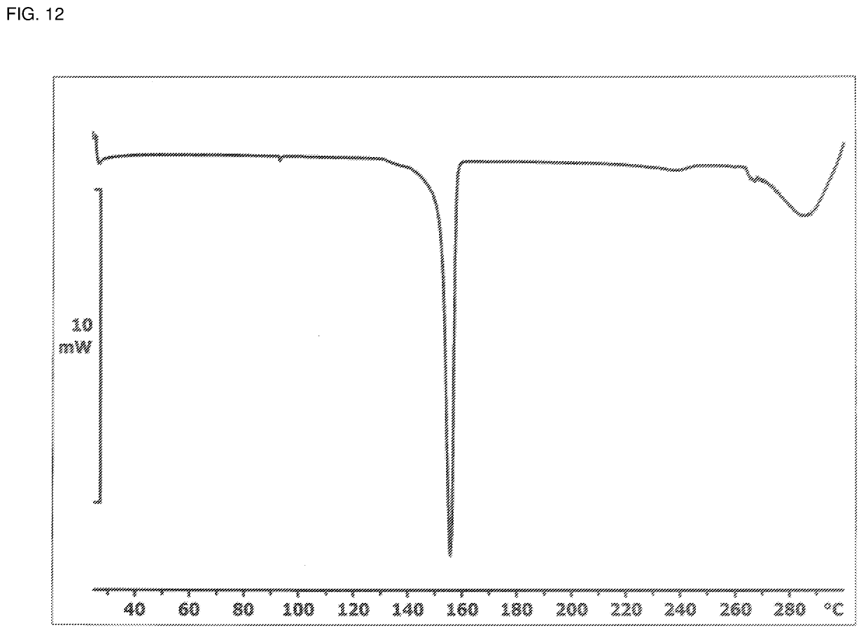

Depending on the manner in which the crystalline forms are prepared, the methodology and instrument used for DSC analysis, it is understood that peaks corresponding with thermal events in a DSC thermogram may vary between ±2° C. from the values observed in the representative DSC thermograms provided in FIGS. 12 to 22 and described herein. Such variations are known and understood by a person of skill in the art, and any such variations do not depart from the invention disclosed herein.

As used herein, the term ‘crystalline form’ refers to a substance with a particular arrangement of molecular components in its crystal lattice, and which may be identified by physical characterization methods such as PXRD and/or DSC.

As used herein, the term “room temperature” refers to a temperature in the range of 20° C. to 25° C.

When describing the embodiments of the present invention there may be a common variance to a given temperature or time that would be understood or expected by the person skilled in the art to provide substantially the same result. For example, when reference is made to a particular temperature, it is to be understood by the person skilled in the art that there is an allowable variance of ±5° C. associated with that temperature. When reference is made to a particular time, it is to be understood that there is an allowable variance of ±10 minutes when the time is one or two hours, and ±1 hour when longer periods of time are referenced.

In a first embodiment of the present invention, there is provided a new salt of zuclomiphene, zuclomiphene sulphate Form APO-I, wherein the molar ratio of zuclomiphene to sulphuric acid is approximately 1:1.

Zuclomiphene sulphate Form APO-I can be characterized by a PXRD diffractogram comprising, among other peaks, characteristic peaks, expressed in degrees 2θ (±0.2°), at 5.2°, 14.6° and 20.6°. Preferably, the PXRD diffractogram further comprises at least three peaks, expressed in degrees 2θ (±0.2°), selected from the group consisting of 10.0°, 10.3°, 12.2°, 12.4°, 12.9°, 15.1°, 16.0°, 17.4°, 22.2° and 22.9°. More preferably, the PXRD diffractogram further comprises peaks, expressed in degrees 2θ (±0.2°), at 10.0°, 10.3°, 12.2°, 12.4°, 12.9°, 15.1°, 16.0°, 17.4°, 22.2° and 22.9°. PXRD studies of uncapped samples of zuclomiphene sulphate Form APO-I maintained in a 40 ° C./75% RH stability chamber for at least 4 weeks showed that no change in the crystalline form occurred.

An illustrative PXRD diffractogram of zuclomiphene sulphate Form APO-I, as prepared in Example 1, is shown in FIG. 1. A peak listing, comprising representative peaks from the PXRD diffractogram in FIG. 1, and their relative intensities, is provided in Table 1. Although illustrative of the PXRD diffractogram that is provided for the zuclomiphene sulphate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 1
Relative peak intensities of zuclomiphene
sulphate Form APO-I from FIG. 1

| Angle (2θ) | Relative intensity (%) |
|---|---|
| 5.17 | 13.0 |
| 9.98 | 8.4 |
| 10.27 | 9.8 |
| 12.19 | 27.6 |
| 12.43 | 29.8 |
| 12.88 | 22.0 |
| 14.64 | 28.7 |
| 15.13 | 29.6 |
| 16.01 | 15.4 |
| 17.39 | 25.0 |
| 20.63 | 100.0 |
| 22.22 | 16.3 |
| 22.91 | 22.8 |
| 23.62 | 12.6 |
| 24.41 | 14.7 |
| 25.08 | 12.2 |

An illustrative DSC thermogram of zuclomiphene sulphate Form APO-I is shown in FIG. 12. The DSC thermogram may be further characterized by an endothermic peak with a peak onset at approximately 152° C. and a peak maximum at approximately 155° C.

In a second embodiment of the present invention, there is provided a new salt of zuclomiphene, zuclomiphene phosphate Form APO-I, wherein the molar ratio of zuclomiphene to phosphoric acid is approximately 1:1.

Zuclomiphene phosphate Form APO-I can be characterized by a PXRD diffractogram comprising, among other peaks, characteristic peaks, expressed in degrees 2θ (±0.2°), at 4.5°, 9.0° and 19.3°. Preferably, the PXRD diffractogram further comprises at least three peaks, expressed in degrees 2θ (±0.2°), selected from the group consisting of 11.0°, 12.5°, 13.5°, 13.8°, 15.0°, 17.7°, 19.3°, 20.6°, 22.3° and 24.2°. More preferably, the PXRD diffractogram further comprises peaks, expressed in degrees 2θ (±0.2°), at 11.0°, 12.5°, 13.5°, 13.8°, 15.0°, 17.7°, 19.3°, 20.6°, 22.3° and 24.2°. PXRD studies of uncapped samples of zuclomiphene phosphate Form APO-I maintained in a 40° C./75% RH stability chamber for at least 4 weeks showed that no change in the crystalline form occurred.

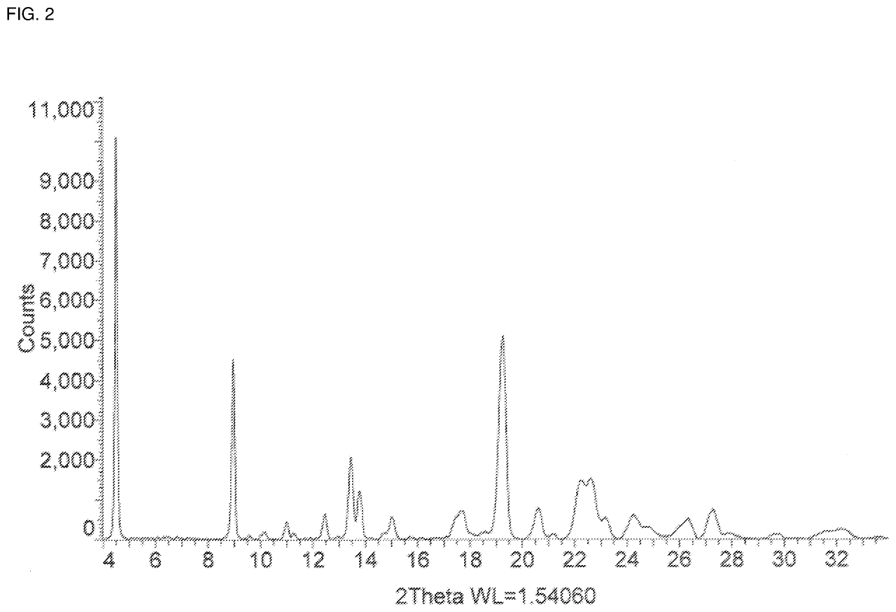

An illustrative PXRD diffractogram of zuclomiphene phosphate Form APO-I, as prepared in Example 2, is shown in FIG. 2. A peak listing, comprising representative peaks from the PXRD diffractogram in FIG. 2, and their relative intensities, is provided in Table 2. Although illustrative of the PXRD diffractogram that is provided for the zuclomiphene phosphate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 2
Relative peak intensities of zuclomiphene
phosphate Form APO-I from FIG. 2

| Angle (2θ) | Relative intensity (%) |
|---|---|
| 4.51 | 100.0 |
| 8.96 | 48.4 |
| 11.00 | 5.1 |
| 12.46 | 7.2 |
| 13.45 | 22.4 |
| 13.78 | 13.1 |
| 15.00 | 6.2 |
| 17.69 | 7.8 |
| 19.26 | 54.9 |
| 20.62 | 8.6 |
| 22.27 | 15.7 |
| 22.61 | 16.6 |
| 24.24 | 6.8 |

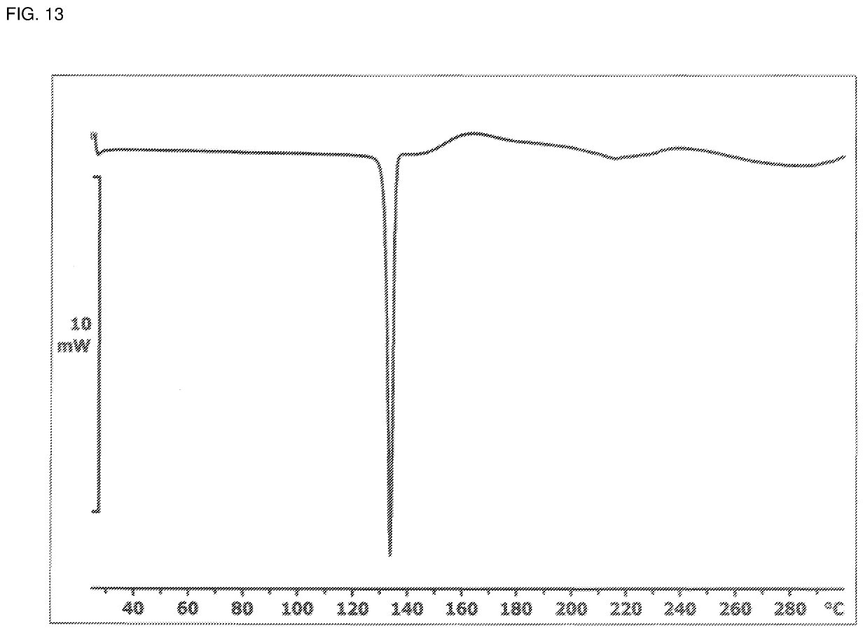

An illustrative DSC thermogram of zuclomiphene phosphate Form APO-I is shown in FIG. 13. The DSC thermogram may be further characterized by an endothermic peak with a peak onset at approximately 131° C. and a peak maximum at approximately 133° C.

In a third embodiment of the present invention, there is provided a new salt of zuclomiphene, zuclomiphene succinate Form APO-I, wherein the molar ratio of zuclomiphene to succinic acid is approximately 1:1.

Zuclomiphene succinate Form APO-I can be characterized by a PXRD diffractogram comprising, among other peaks, characteristic peaks, expressed in degrees 2θ (±0.2°), at 5.5°, 10.1° and 17.3°. Preferably, the PXRD diffractogram further comprises at least three peaks, expressed in degrees 2θ (±0.2°), selected from the group consisting of 11.8°, 13.9°, 15.2°, 18.6°, 19.7°, 20.1°, 21.4°, 22.1°, 22.9° and 23.7°. More preferably, the PXRD diffractogram further comprises peaks, expressed in degrees 2θ (±0.2°), at 11.8°, 13.9°, 15.2°, 18.6°, 19.7°, 20.1°, 21.4°, 22.1°, 22.9° and 23.7°. PXRD studies of uncapped samples of zuclomiphene succinate Form APO-I maintained in a 40° C./75% RH stability chamber for at least 4 weeks showed that no change in the crystalline form occurred.

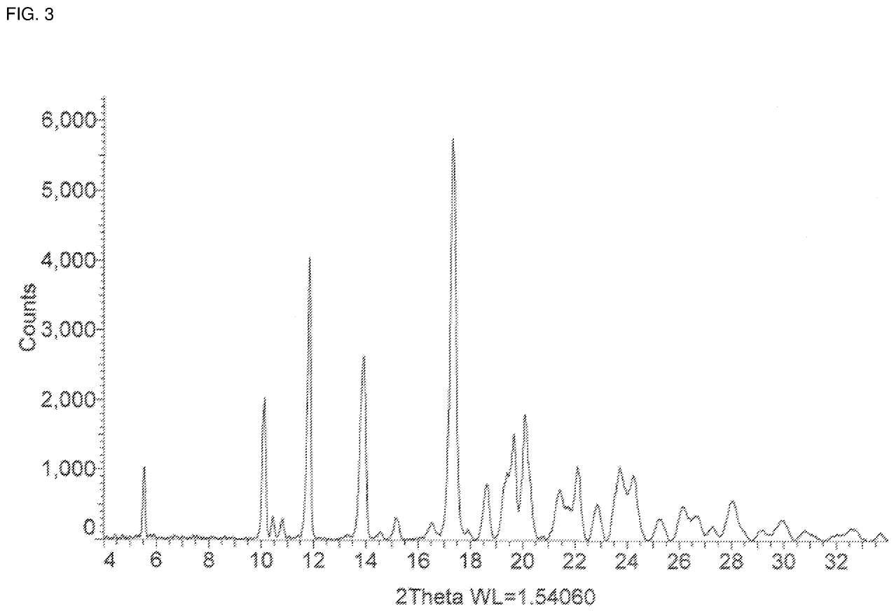

An illustrative PXRD diffractogram of zuclomiphene succinate Form APO-I, as prepared in Example 3, is shown in FIG. 3. A peak listing, comprising representative peaks from the PXRD diffractogram in FIG. 3, and their relative intensities, is provided in Table 3. Although illustrative of the PXRD diffractogram that is provided for the zuclomiphene succinate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 3
Relative peak intensities of zuclomiphene
succinate Form APO-I from FIG. 3

| Angle (2θ) | Relative intensity (%) |
|---|---|
| 5.53 | 16.1 |
| 10.12 | 34.7 |
| 10.46 | 5.8 |
| 10.81 | 5.1 |
| 11.83 | 70.9 |
| 13.91 | 44.2 |
| 15.15 | 5.5 |
| 17.31 | 100.0 |
| 18.64 | 13.1 |
| 19.68 | 26.8 |
| 20.10 | 31.5 |
| 21.43 | 12.1 |
| 22.10 | 18.3 |
| 22.86 | 9.2 |
| 23.72 | 18.3 |

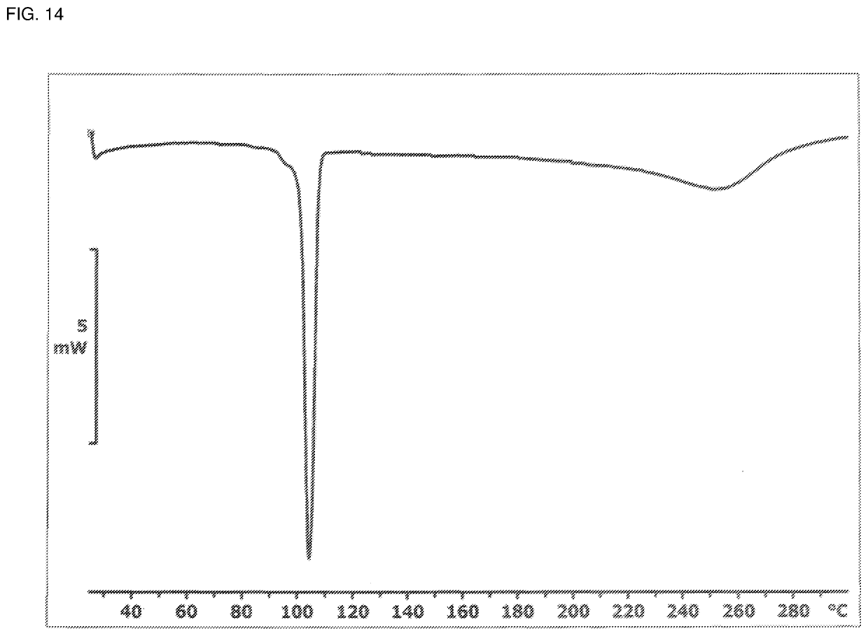

An illustrative DSC thermogram of zuclomiphene succinate Form APO-I is shown in FIG. 14. The DSC thermogram may be further characterized by an endothermic peak with a peak onset at approximately 101° C. and a peak maximum at approximately 104° C.

In a fourth embodiment of the present invention, there is provided a new salt of zuclomiphene, zuclomiphene L-tartrate Form APO-I, wherein the molar ratio of zuclomiphene to L-tartaric acid is approximately 1:1.

Zuclomiphene L-tartrate Form APO-I can be characterized by a PXRD diffractogram comprising, among other peaks, characteristic peaks, expressed in degrees 2θ (±0.2°), at 6.0°, 9.0° and 12.0°. Preferably, the PXRD diffractogram further comprises at least three peaks, expressed in degrees 2θ (±0.2°), selected from the group consisting of 11.4°, 13.7°, 14.9°, 15.9°, 17.4°, 18.3°, 18.9°, 20.4°, 20.9° and 22.4°. More preferably, the PXRD diffractogram further comprises peaks, expressed in degrees 2θ (±0.2°), at 11.4°, 13.7°, 14.9°, 15.9°, 17.4°, 18.3°, 18.9°, 20.4°, 20.9° and 22.4°. PXRD studies of uncapped samples of zuclomiphene L-tartrate Form APO-I maintained in a 40° C./75% RH stability chamber for at least 4 weeks showed that no change in the crystalline form occurred.

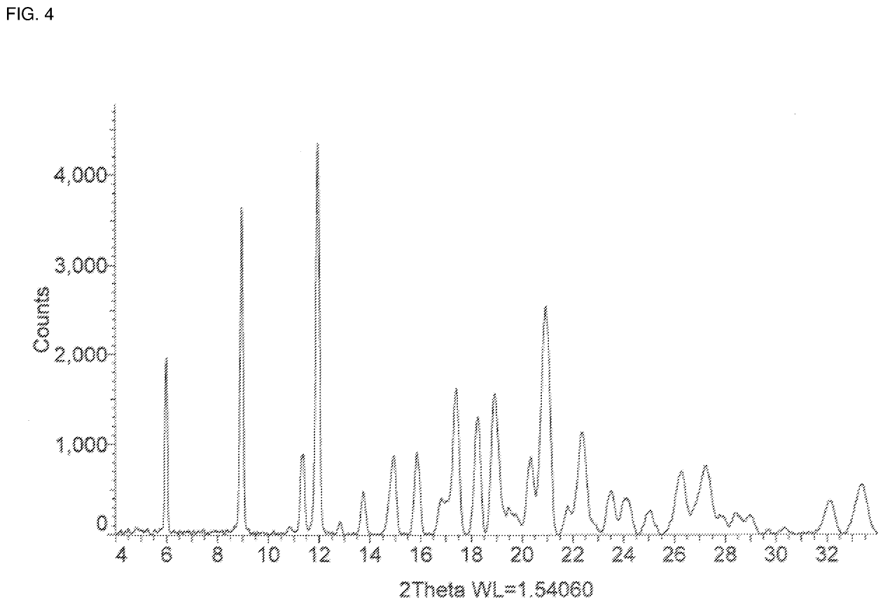

An illustrative PXRD diffractogram of zuclomiphene L-tartrate Form APO-I, as prepared in Example 4, is shown in FIG. 4. A peak listing, comprising representative peaks from the PXRD diffractogram in FIG. 4, and their relative intensities, is provided in Table 4. Although illustrative of the PXRD diffractogram that is provided for the zuclomiphene L-tartrate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 4
Relative peak intensities of zuclomiphene
L-tartrate Form APO-I from FIG. 4

| Angle (2θ) | Relative intensity (%) |
|---|---|
| 5.99 | 41.8 |
| 8.97 | 81.2 |
| 11.37 | 20.2 |
| 11.96 | 100.0 |
| 13.74 | 11.0 |
| 14.94 | 19.4 |
| 15.86 | 20.7 |
| 17.41 | 36.8 |
| 18.26 | 29.4 |
| 18.93 | 35.0 |
| 20.35 | 19.7 |
| 20.94 | 58.5 |
| 22.37 | 25.5 |
| 23.50 | 11.2 |
| 24.12 | 9.3 |

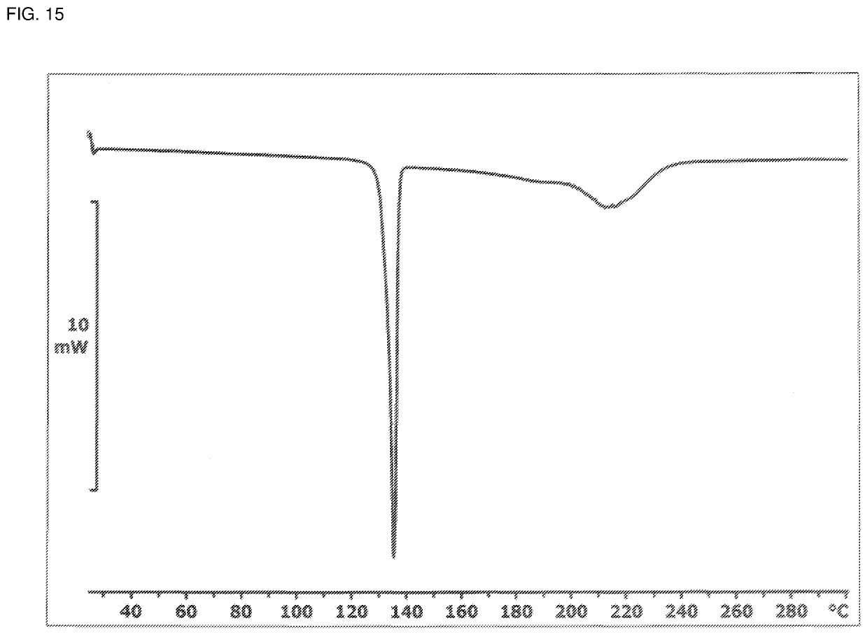

An illustrative DSC thermogram of zuclomiphene L-tartrate Form APO-I is shown in FIG. 15. The DSC thermogram may be further characterized by an endothermic peak with a peak onset at approximately 132° C. and a peak maximum at approximately 135° C.

In a fifth embodiment of the present invention, there is provided a new salt of zuclomiphene, zuclomiphene tosylate Form APO-I, wherein the molar ratio of zuclomiphene to p-toluenesulfonic acid is approximately 1:1.

Zuclomiphene tosylate Form APO-I can be characterized by a PXRD diffractogram comprising, among other peaks, characteristic peaks, expressed in degrees 2θ (±0.2°), at 5.6°, 11.1° and 18.1°. Preferably, the PXRD diffractogram further comprises at least three peaks, expressed in degrees 2θ (±0.2°), selected from the group consisting of 5.0°, 8.6°, 10.4°, 13.3°, 14.0°, 16.8°, 19.3°, 21.9°, 22.8° and 24.6°. More preferably, the PXRD diffractogram further comprises peaks, expressed in degrees 2θ (±0.2°), at 5.0°, 8.6°, 10.4°, 13.3°, 14.0°, 16.8°, 19.3°, 21.9°, 22.8° and 24.6°. PXRD studies of uncapped samples of zuclomiphene tosylate Form APO-I maintained in a 40° C./75% RH stability chamber for at least 4 weeks showed that no change in the crystalline form occurred.

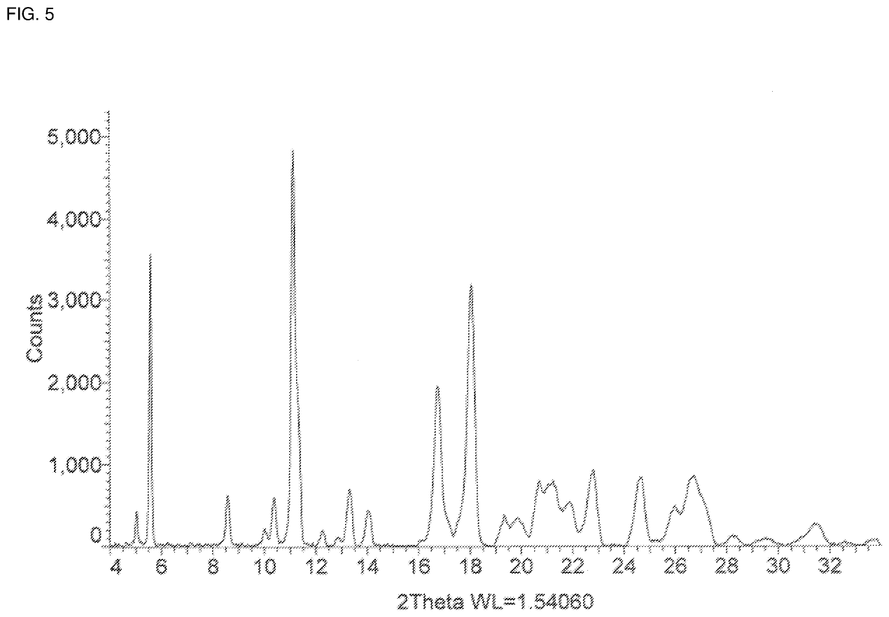

An illustrative PXRD diffractogram of zuclomiphene tosylate Form APO-I, as prepared in Example 5, is shown in FIG. 5. A peak listing, comprising representative peaks from the PXRD diffractogram in FIG. 5, and their relative intensities, is provided in Table 5. Although illustrative of the PXRD diffractogram that is provided for the zuclomiphene tosylate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 5
Relative peak intensities of zuclomiphene
tosylate Form APO-I from FIG. 5

| Angle (2θ) | Relative intensity (%) |
|---|---|
| 5.03 | 7.8 |
| 5.58 | 68.7 |
| 8.58 | 12.9 |
| 10.39 | 12.4 |
| 11.12 | 100.0 |
| 13.31 | 14.4 |
| 14.03 | 9.0 |
| 16.75 | 39.8 |
| 18.06 | 65.3 |
| 19.34 | 7.9 |
| 20.69 | 16.7 |
| 21.22 | 16.6 |
| 21.87 | 11.3 |
| 22.79 | 19.3 |
| 24.65 | 17.5 |

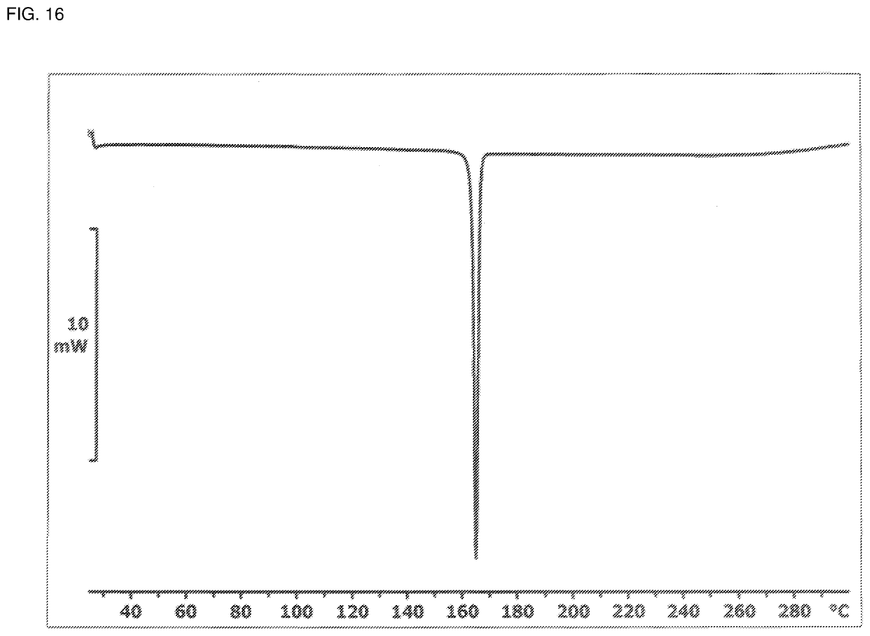

An illustrative DSC thermogram of zuclomiphene tosylate Form APO-I is shown in FIG. 16. The DSC thermogram may be further characterized by an endothermic peak with a peak onset at approximately 163° C. and a peak maximum at approximately 164° C.

In a sixth embodiment of the present invention, there is provided a new salt of zuclomiphene, zuclomiphene L-malate Form APO-I, wherein the molar ratio of zuclomiphene to L-malic acid is approximately 1:1.

Zuclomiphene L-malate Form APO-I can be characterized by a PXRD diffractogram comprising, among other peaks, characteristic peaks, expressed in degrees 2θ (±0.2°), at 6.4°, 12.8° and 22.5°. Preferably, the PXRD diffractogram further comprises at least three peaks, expressed in degrees 2θ (±0.2°), selected from the group consisting of 9.6°, 10.1°, 12.0°, 14.0°, 15.6°, 16.0°, 16.6°, 18.1°, 18.7° and 19.0°. More preferably, the PXRD diffractogram further comprises peaks, expressed in degrees 2θ (±0.2°), at 9.6°, 10.1°, 12.0°, 14.0°, 15.6°, 16.0°, 16.6°, 18.1°, 18.7° and 19.0°. PXRD studies of uncapped samples of zuclomiphene L-malate Form APO-I maintained in a 40° C./75% RH stability chamber for at least 4 weeks showed that no change in the crystalline form occurred.

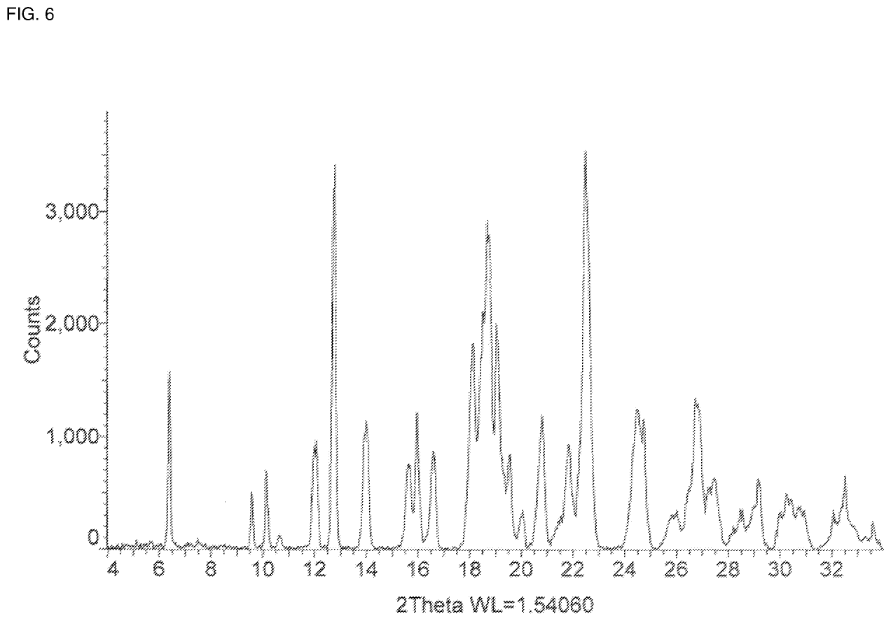

An illustrative PXRD diffractogram of zuclomiphene L-malate Form APO-I, as prepared in Example 6, is shown in FIG. 6. A peak listing, comprising representative peaks from the PXRD in FIG. 6, and their relative intensities, is provided in Table 6. Although illustrative of the PXRD diffractogram that is provided for the zuclomiphene L-malate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 6
Relative peak intensities of zuclomiphene
L-malate Form APO-I from FIG. 6

| Angle (2θ) | Relative intensity (%) |
|---|---|
| 6.40 | 40.1 |
| 9.58 | 13.3 |
| 10.15 | 17.4 |
| 12.02 | 23.3 |
| 12.78 | 92.2 |
| 14.00 | 31.3 |
| 15.63 | 20.1 |
| 15.96 | 36.5 |
| 16.60 | 24.1 |
| 18.12 | 49.4 |
| 18.68 | 80.7 |
| 19.05 | 53.9 |
| 20.80 | 32.7 |
| 21.83 | 25.0 |
| 22.47 | 100.0 |
| 24.49 | 33.8 |

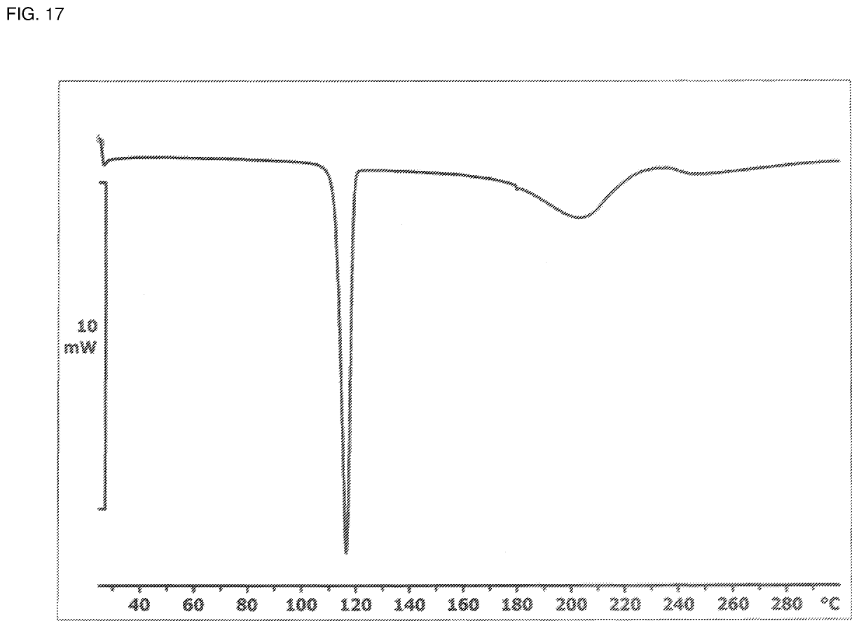

An illustrative DSC thermogram of zuclomiphene L-malate Form APO-I is shown in FIG. 17. The DSC thermogram may be further characterized by an endothermic peak with a peak onset at approximately 112° C. and a peak maximum at approximately 116° C.

In a seventh embodiment of the present invention, there is provided a new salt of zuclomiphene, zuclomiphene maleate Form APO-I, wherein the molar ratio of zuclomiphene to maleic acid is approximately 1:1.

Zuclomiphene maleate Form APO-I can be characterized by a PXRD diffractogram comprising, among other peaks, characteristic peaks, expressed in degrees 2θ (±0.2°), at 6.6°, 13.2° and 20.5°. Preferably, the PXRD diffractogram further comprises at least three peaks, expressed in degrees 2θ (±0.2°), selected from the group consisting of 12.1°, 14.2°, 15.0°, 16.1°, 16.5°, 17.8°, 18.2°, 19.5°, 19.8° and 22.3°. More preferably, the PXRD diffractogram further comprises peaks, expressed in degrees 2θ (±0.2°), at 12.1°, 14.2°, 15.0°, 16.1°, 16.5°, 17.8°, 18.2°, 19.5°, 19.8° and 22.3°.

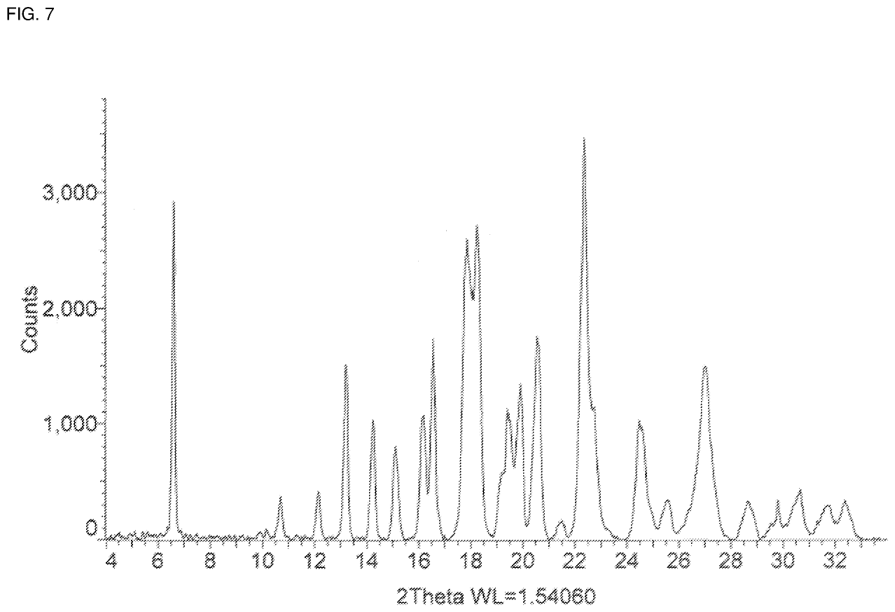

An illustrative PXRD diffractogram of zuclomiphene maleate Form APO-I, as prepared in Example 7, is shown in FIG. 7. A peak listing, comprising representative peaks from the PXRD in FIG. 7, and their relative intensities, is provided in Table 7. Although illustrative of the PXRD diffractogram that is provided for the zuclomiphene maleate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 7
Relative peak intensities of zuclomiphene
maleate Form APO-I from FIG. 7

| Angle (2θ) | Relative intensity (%) |
|---|---|
| 6.59 | 100.0 |
| 12.11 | 9.3 |
| 13.16 | 35.2 |
| 14.19 | 12.3 |
| 15.05 | 12.1 |
| 16.10 | 17.2 |
| 16.45 | 25.0 |
| 17.81 | 43.4 |
| 18.18 | 45.6 |
| 19.49 | 15.6 |
| 19.80 | 20.7 |
| 20.46 | 24.0 |
| 22.28 | 37.2 |
| 24.40 | 15.0 |

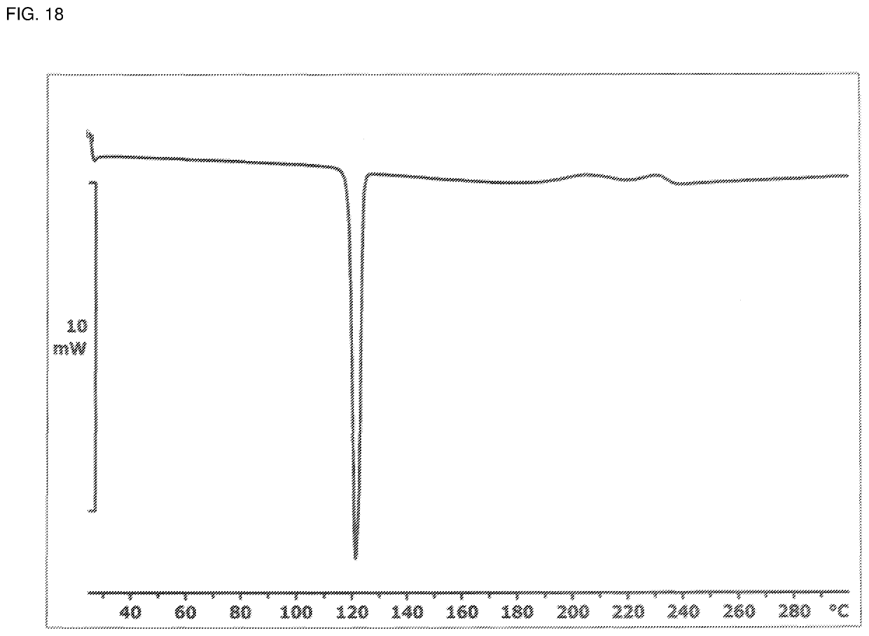

An illustrative DSC thermogram of zuclomiphene maleate Form APO-I is shown in FIG. 18. The DSC thermogram may be further characterized by an endothermic peak with a peak onset at approximately 119° C. and a peak maximum at approximately 121° C.

In an eighth embodiment of the present invention, there is provided a new salt of zuclomiphene, zuclomiphene malonate Form APO-I, wherein the molar ratio of zuclomiphene to malonic acid is approximately 1:1.

Zuclomiphene malonate Form APO-I can be characterized by a PXRD diffractogram comprising, among other peaks, characteristic peaks, expressed in degrees 2θ (±0.2°), at 6.8°, 13.6° and 18.2°. Preferably, the PXRD diffractogram further comprises at least three peaks, expressed in degrees 2θ (±0.2°), selected from the group consisting of 11.3°, 15.6°, 17.0°, 19.9°, 20.4°, 21.4°, 22.0°, 22.9°, 23.7° and 26.1°. More preferably, the PXRD diffractogram further comprises peaks, expressed in degrees 2θ (±0.2°), at 11.3°, 15.6°, 17.0°, 19.9°, 20.4°, 21.4°, 22.0°, 22.9°, 23.7° and 26.1°.

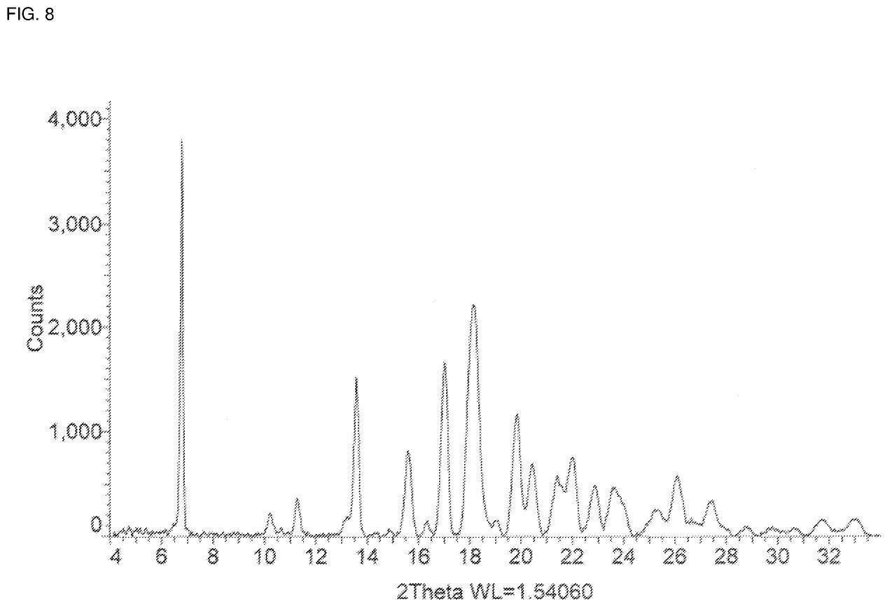

An illustrative PXRD diffractogram of zuclomiphene malonate Form APO-I, as prepared in Example 8, is shown in FIG. 8. A peak listing, comprising representative peaks from the PXRD in FIG. 8, and their relative intensities, is provided in Table 8. Although illustrative of the PXRD diffractogram that is provided for the zuclomiphene malonate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 8
Relative peak intensities of zuclomiphene
malonate Form APO-I from FIG. 8

| Angle (2θ) | Relative intensity (%) |
|---|---|
| 6.80 | 100.0 |
| 11.28 | 10.0 |
| 13.60 | 42.8 |
| 15.61 | 22.2 |
| 17.03 | 46.3 |
| 18.16 | 60.8 |
| 19.85 | 32.6 |
| 20.44 | 19.7 |
| 21.44 | 16.4 |
| 22.02 | 20.5 |
| 22.88 | 13.6 |
| 23.65 | 13.2 |
| 26.09 | 16.2 |

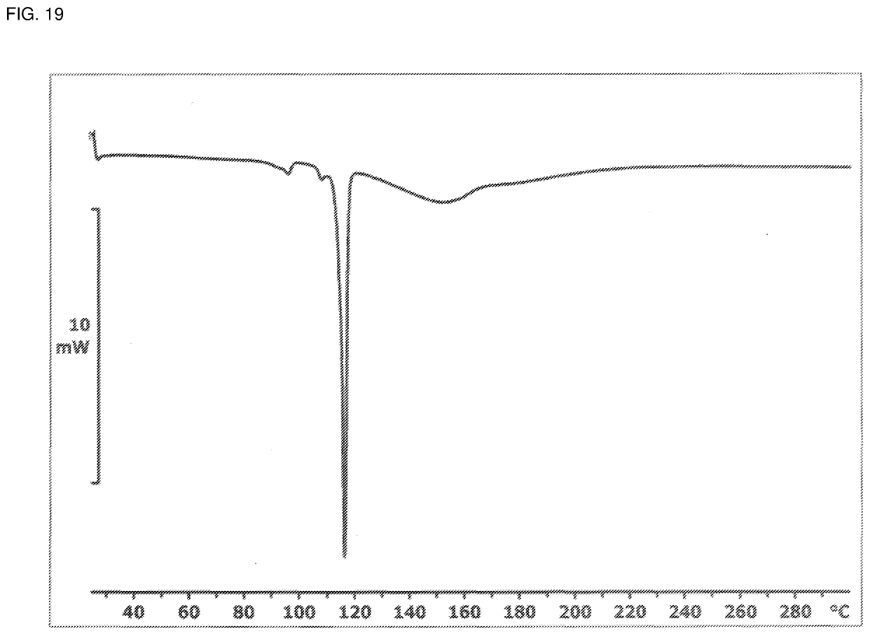

An illustrative DSC thermogram of zuclomiphene malonate Form APO-I is shown in FIG. 19. The DSC thermogram may be further characterized by an endothermic peak with a peak onset at approximately 114° C. and a peak maximum at approximately 116° C.

In a ninth embodiment of the present invention, there is provided a new salt of zuclomiphene, zuclomiphene fumarate Form APO-I, wherein the molar ratio of zuclomiphene to fumaric acid is approximately 1:1.

Zuclomiphene fumarate Form APO-I can be characterized by a PXRD diffractogram comprising, among other peaks, characteristic peaks, expressed in degrees 2θ (±0.2°), at 6.9°, 13.9° and 17.9°. Preferably, the PXRD diffractogram further comprises at least three peaks, expressed in degrees 2θ (±0.2°), selected from the group consisting of 10.0°, 10.5°, 15.6°, 16.3°, 17.4°, 19.3° and 21.1°. More preferably, the PXRD diffractogram further comprises peaks, expressed in degrees 2θ (±0.2°), at 10.0°, 10.5°, 15.6°, 16.3°, 17.4°, 19.3° and 21.1°. PXRD studies of uncapped samples of zuclomiphene fumarate Form APO-I maintained in a 40° C./75% RH stability chamber for at least 4 weeks showed that no change in the crystalline form occurred.

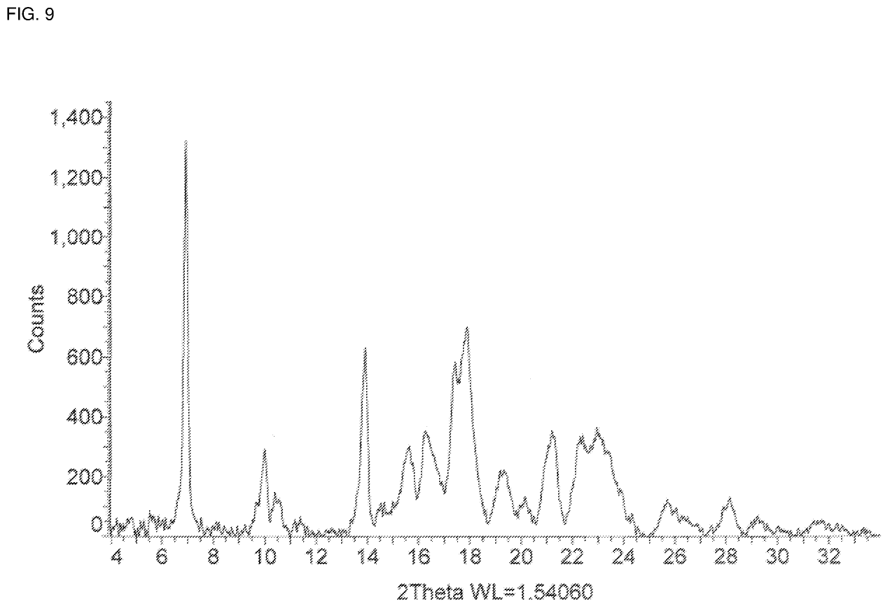

An illustrative PXRD diffractogram of zuclomiphene fumarate Form APO-I, as prepared in Example 9, is shown in FIG. 9. A peak listing, comprising representative peaks from the PXRD in FIG. 9, and their relative intensities, is provided in Table 9. Although illustrative of the PXRD diffractogram that is provided for the zuclomiphene fumarate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 9
Relative peak intensities of zuclomiphene
fumarate Form APO-I from FIG. 9

| Angle (2θ) | Relative intensity (%) |
|---|---|
| 6.94 | 100.0 |
| 9.99 | 22.9 |
| 10.50 | 9.1 |
| 13.92 | 49.8 |
| 15.63 | 23.5 |
| 16.32 | 26.2 |
| 17.43 | 45.9 |
| 17.88 | 54.9 |
| 19.32 | 17.6 |
| 21.14 | 26.3 |
| 22.31 | 26.6 |
| 22.96 | 30.2 |
| 25.70 | 10.7 |

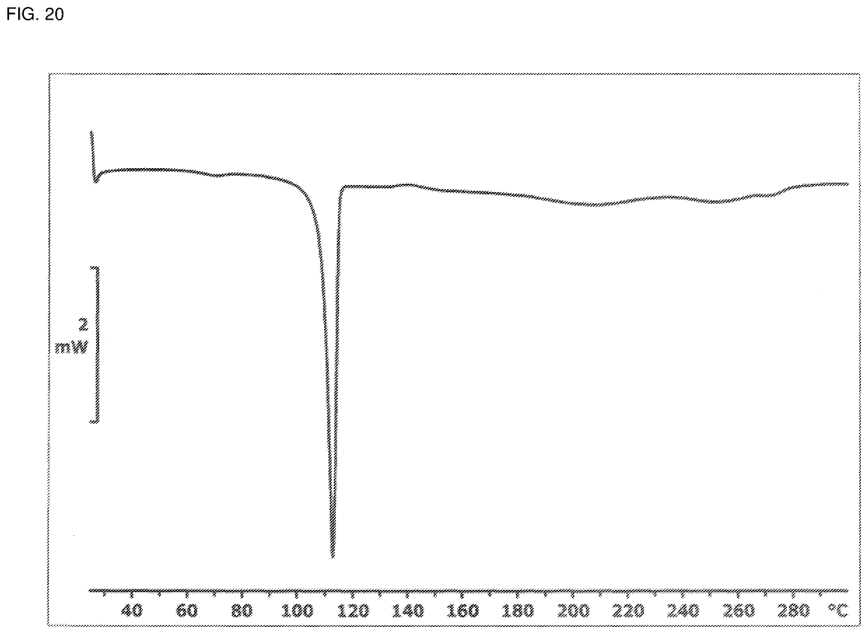

An illustrative DSC thermogram of zuclomiphene fumarate Form APO-I is shown in FIG. 20. The DSC thermogram may be further characterized by an endothermic peak with a peak onset at approximately 110° C. and a peak maximum at approximately 113° C.

In a tenth embodiment of the present invention, there is provided a new salt of zuclomiphene, zuclomiphene glycolate Form APO-I, wherein the molar ratio of zuclomiphene to glycolic acid is approximately 1:1.

Zuclomiphene glycolate Form APO-I can be characterized by a PXRD diffractogram comprising, among other peaks, characteristic peaks, expressed in degrees 2θ (±0.2°), at 6.0°, 9.0° and 18.2°. Preferably, the PXRD diffractogram further comprises at least three peaks, expressed in degrees 2θ (±0.2°), selected from the group consisting of 9.7°, 10.5°, 12.0°, 15.2°, 15.9°, 17.3°, 18.2°, 20.0°, 21.2° and 23.5°. More preferably, the PXRD diffractogram further comprises peaks, expressed in degrees 2θ (±0.2°), at 9.7°, 10.5°, 12.0°, 15.2°, 15.9°, 17.3°, 18.2°, 20.0°, 21.2° and 23.5°.

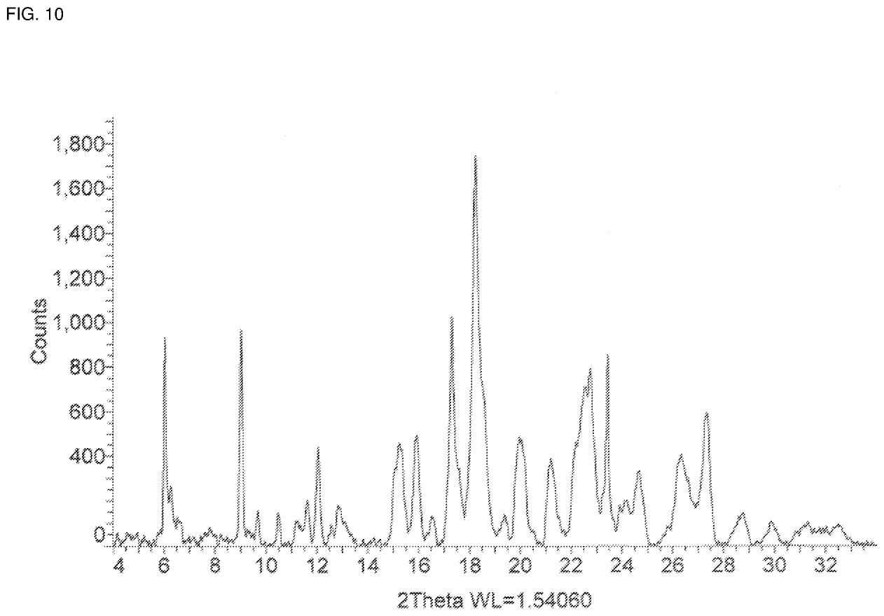

An illustrative PXRD diffractogram of zuclomiphene glycolate Form APO-I, as prepared in Example 10, is shown in FIG. 10. A peak listing, comprising representative peaks from the PXRD in FIG. 10, and their relative intensities, is provided in Table 10. Although illustrative of the PXRD diffractogram that is provided for the zuclomiphene glycolate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 10
Relative peak intensities of zuclomiphene
glycolate Form APO-I from FIG. 10

| Angle (2θ) | Relative intensity (%) |
|---|---|
| 6.03 | 48.8 |
| 9.02 | 53.8 |
| 9.68 | 8.7 |
| 10.47 | 7.8 |
| 11.62 | 11.5 |
| 12.04 | 24.6 |
| 15.24 | 25.5 |
| 15.92 | 28.4 |
| 17.31 | 62.1 |
| 18.24 | 100.0 |
| 19.99 | 25.1 |
| 21.20 | 22.5 |
| 22.76 | 44.6 |
| 23.45 | 53.7 |
| 24.66 | 18.2 |

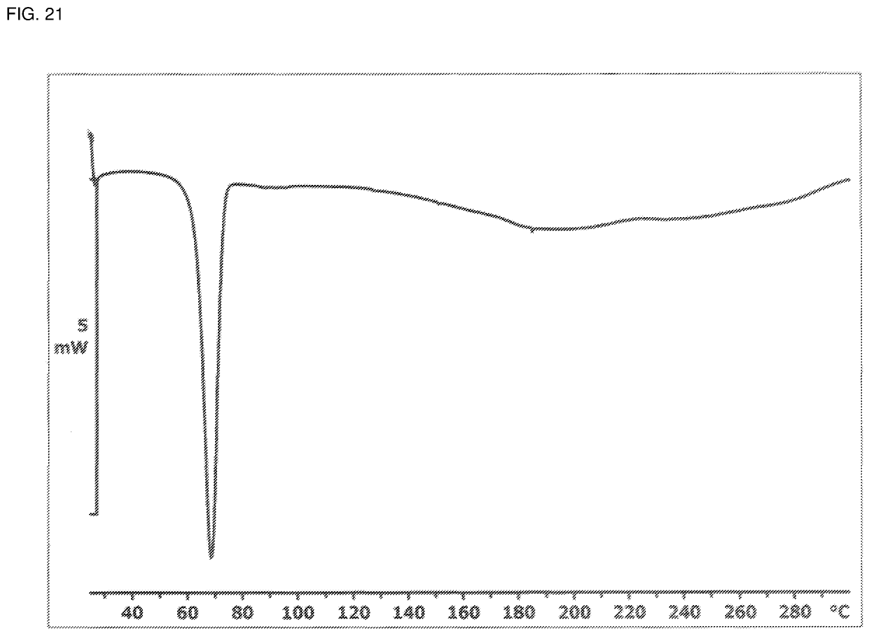

An illustrative DSC thermogram of zuclomiphene glycolate Form APO-I is shown in FIG. 21. The DSC thermogram may be further characterized by an endothermic peak with a peak onset at approximately 63° C. and a peak maximum at approximately 68° C.

In an eleventh embodiment of the present invention, there is provided a new salt of zuclomiphene, zuclomiphene hemi-citrate Form APO-I, wherein the molar ratio of zuclomiphene to citric acid is approximately 1:1.

Zuclomiphene hemi-citrate Form APO-I can be characterized by a PXRD diffractogram comprising, among other peaks, characteristic peaks, expressed in degrees 2θ (±0.2°), at 5.0°, 13.3° and 16.8°. Preferably, the PXRD diffractogram further comprises at least three peaks, expressed in degrees 2θ (±0.2°), selected from the group consisting of 9.5°, 10.9°, 14.6°, 15.7°, 18.2°, 20.2°, 20.9°, 21.6° and 24.0°. More preferably, the PXRD diffractogram further comprises peaks, expressed in degrees 2θ (±0.2°), at 9.5°, 10.9°, 14.6°, 15.7°, 18.2°, 20.2°, 20.9°, 21.6° and 24.0°.

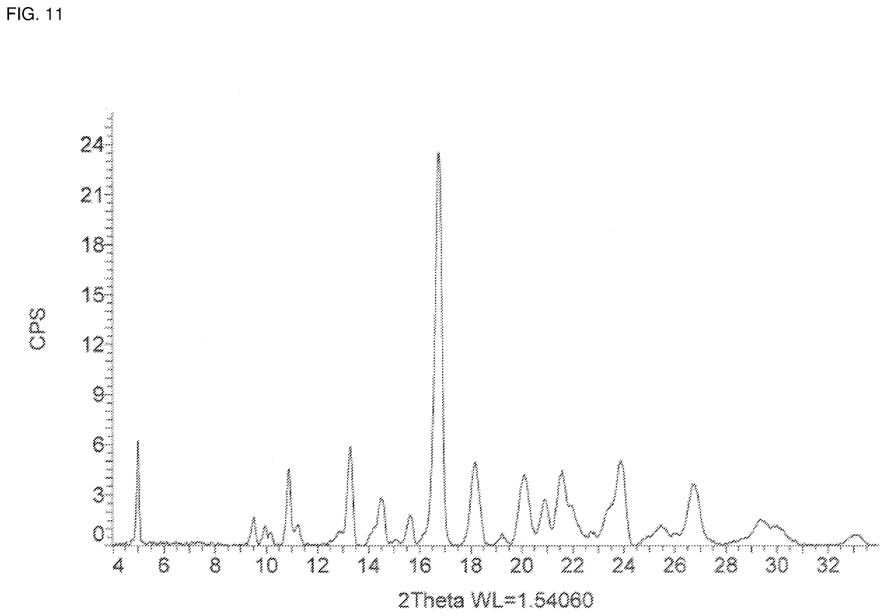

An illustrative PXRD diffractogram of zuclomiphene hemi-citrate Form APO-I, as prepared in Example 11, is shown in FIG. 11. A peak listing, comprising representative peaks from the PXRD in FIG. 11, and their relative intensities, is provided in Table 11. Although illustrative of the PXRD diffractogram that is provided for the zuclomiphene hemi-citrate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 11
Relative peak intensities of zuclomiphene
hemi-citrate Form APO-I from FIG. 11

| Angle (2θ) | Relative intensity (%) |
|---|---|
| 5.02 | 12.3 |
| 9.53 | 8.2 |
| 10.91 | 22.9 |
| 13.31 | 32.8 |
| 14.55 | 15.0 |
| 15.67 | 8.9 |
| 16.79 | 100.0 |
| 18.20 | 21.8 |
| 20.15 | 17.0 |
| 20.90 | 11.9 |
| 21.59 | 18.8 |
| 23.95 | 30.9 |
| 26.80 | 18.7 |

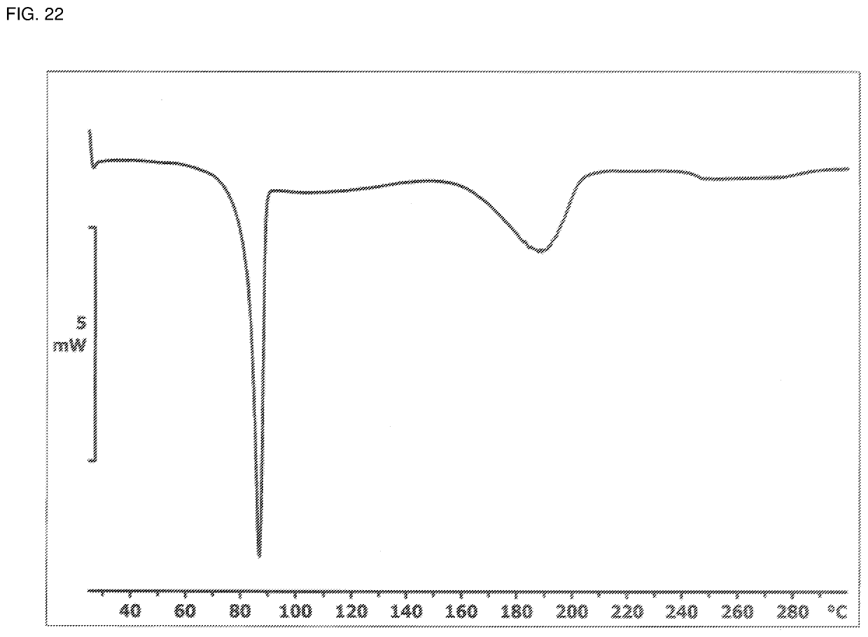

An illustrative DSC thermogram of zuclomiphene hemi-citrate Form APO-I is shown in FIG. 22. The DSC thermogram may be further characterized by an endothermic peak with a peak onset at approximately 82° C. and a peak maximum at approximately 86° C.

In a twelfth embodiment of the invention, there is provided a pharmaceutical composition comprising zuclomiphene sulphate, zuclomiphene phosphate, zuclomiphene succinate, zuclomiphene L-tartrate, zuclomiphene tosylate, zuclomiphene L-malate, zuclomiphene maleate, zuclomiphene malonate, zuclomiphene fumarate, zuclomiphene glycolate, or zuclomiphene hemi-citrate, with one or more pharmaceutically acceptable excipients. Preferably, the pharmaceutical composition is a solid dosage form suitable for oral administration, such as a capsule, tablet, pill, powder or granulate. Most preferably, the pharmaceutical composition is a tablet or a capsule. Preferably, the pharmaceutical composition provides a dose of zuclomiphene sulphate, zuclomiphene phosphate, zuclomiphene succinate, zuclomiphene L-tartrate, zuclomiphene tosylate, zuclomiphene L-malate, zuclomiphene maleate, zuclomiphene malonate, zuclomiphene fumarate, zuclomiphene glycolate, or zuclomiphene hemi-citrate that is equivalent to the 1 to 80 mg of zuclomiphene citrate that is described as a dosage range in U.S. Pat. No. 9,913,815 B2. More preferably, the pharmaceutical composition provides a dose of zuclomiphene sulphate, zuclomiphene phosphate, zuclomiphene succinate, zuclomiphene L-tartrate, zuclomiphene tosylate, zuclomiphene L-malate, zuclomiphene maleate, zuclomiphene malonate, zuclomiphene fumarate, zuclomiphene glycolate, or zuclomiphene hemi-citrate that is equivalent to the 50 mg of zuclomiphene citrate that was demonstrated in interim Phase 2 clinical trial results to provide a statistically significant decrease in moderate to severe hot flashes from baseline (Veru Inc. “Veru Announces Positive Top-Line Interim Data from Phase 2 Clinical Trial of Zuclomiphene to Treat Hot Flashes in Men with Prostate Cancer on Androgen Deprivation Therapy.” Veru Inc. press release, Jan. 12, 2020. On the Veru Inc. website. https://verupharma.com/news/, accessed Jan. 16, 2020).

Suitable pharmaceutically acceptable excipients are preferably inert with respect to the zuclomiphene salts of the present invention, and may include, for example, one or more excipients selected from binders such as lactose, starches, modified starches, sugars, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone, gelatine, polyvinylpyrrolidone (PVP) and sodium alginate; fillers or diluents such as lactose, sugar, starches, modified starches, mannitol, sorbitol, inorganic salts, cellulose derivatives (e.g., microcrystalline cellulose, cellulose), calcium sulphate, xylitol and lactitol; disintegrants such as croscarmellose sodium, crospovidone, polyvinylpyrrolidone, sodium starch glycollate, corn starch, microcrystalline cellulose, hydroxypropyl methylcellulose and hydroxypropyl cellulose; lubricants such as magnesium stearate, magnesium lauryl stearate, sodium stearyl fumarate, stearic acid, calcium stearate, zinc stearate, potassium benzoate, sodium benzoate, myristic acid, palmitic acid, mineral oil, hydrogenated castor oil, medium-chain triglycerides, poloxamer, polyethylene glycol and talc; and dispersants or solubility enhancing agents, such cyclodextrins, glyceryl monostearate, hypromellose, meglumine, Poloxamer, polyoxyethylene castor oil derivatives, polyoxyethylene stearates, polyoxylglycerides, povidone, and stearic acid. Other excipients including preservatives, stabilisers, anti-oxidants, silica flow conditioners, antiadherents or glidants may be added as required. Other suitable excipients and the preparation of solid oral dosage forms are well known to person of skill in the art, and is described generally, for example, in Remington The Science and Practice of Pharmacy 21st Edition (Lippincott Williams & Wilkins: Philadelphia; 2006; Chapter 45).

Optionally, when the pharmaceutical compositions are solid dosage forms, the solid dosage forms may be prepared with coatings, such as enteric coatings and extended release coatings, using standard pharmaceutical coatings. Such coatings, and their application, are well known to persons skilled in the art, and are described, for example, in Remington The Science and Practice of Pharmacy 21st Edition (Lippincott Williams & Wilkins: Philadelphia; 2006; Chapter 46).

### EXAMPLES

The following non-limiting examples are illustrative of some of the aspects and embodiments of the invention described herein.

### PXRD Analysis:

PXRD diffractograms were recorded on a Bruker-AXS D8 Discover powder X-ray diffractometer (Bruker-AXS, Karlsruhe, Germany). The X-rays were generated using an Incoatec Micro-focus X-ray source (IpSTube: Cu tube with λ=1.54060 Å) with a voltage of 50 kV and current of 1.00 mA, using a micro mask 0.3 mm plug-in pinhole microslit and a short UBC 45 mm long collimator with a 0.3 mm diameter. For each sample, one frame was collected using a still scan with a Pilatus 3R-100K-A area detector at the distance of 154.72 mm from the sample. Raw data were evaluated using the program DIFFRAC.EVA (Bruker-AXS, Karlsruhe, Germany).

### Differential Scanning Calorimetry Analysis

DSC thermograms were collected on a Mettler-Toledo 821e instrument. Samples (1.4 to 3 mg) were weighed into individual 40 μL aluminum pans and were crimped closed with an aluminum lid having a 50 μm perforation. The samples were analyzed under a flow of nitrogen (50±5 mL/min) at a scan rate of 10° C./minute between 25° C. and 300° C.

### Intrinsic Dissolution Testing

Intrinsic dissolution rate (IDR) measurements were performed using a Wood apparatus (Pharma Test PT-DT8 instrument) having a bath temperature of 37° C. Samples were prepared by compressing 200-400 mg samples at 1.5 metric tons for 1 minute. A dissolution medium consisting of 900 mL distilled water, and rotation speed of 50 rpm, was used for each experiment.

### Example 1

### Preparation of Zuclomiphene Sulphate Form APO-I

A mixture of zuclomiphene (100 mg, 0.25 mmol) and sulphuric acid (14 μL, 0.26 mmol) in MeOH (0.14 mL) and ethyl acetate (6 mL) was stirred at room temperature for 2 hours. The mixture was allowed to stand to crystallize overnight. The solvent was decanted off and the white crystalline solid was washed with n-heptane. The solid was recrystallized in minimal hot ethyl acetate to afford zuclomiphene sulphate Form APO-I. The PXRD and DSC thermogram of a sample prepared by this method are shown in FIG. 1 and FIG. 12, respectively.

¹H NMR (DMSO-d₆, 400 MHz): δ1.24 (t, J=7.2 Hz, 6H), 3.24 (m, 4H), 3.55 (br s, 2H), 4.34 (br s, 2H), 6.94-7.04 (m, 4H), 7.12-7.15 (m, 3H), 7.22-7.32 (m, 7H), 9.20 (br s, 1H)

### Example 2

### Preparation of Zuclomiphene Phosphate Form APO-I

A mixture of zuclomiphene (100 mg, 0.25 mmol) and phosphoric acid (14 μL, 0.26 mmol) in MeOH (0.14 mL) and ethyl acetate (6 mL) was stirred at room temperature for 2 hours. The mixture was allowed to stand to crystallize. After 3 days, the solvent was decanted off and the white crystalline solid was washed with n-heptane. The solid was recrystallized in minimal hot ethyl acetate to afford zuclomiphene phosphate Form APO-I. The PXRD and DSC thermogram of a sample prepared by this method are shown in FIG. 2 and FIG. 13, respectively.

¹H NMR (DMSO-d₆, 400 MHz): δ1.06 (m, 6H), 2.78 (br s, 4H), 3.03 (m, 2H), 4.13 (m, 2H), 6.94-6.99 (m, 4H), 7.12-7.14 (m, 3H), 7.21-7.27 (m, 7H).

### Example 3

### Preparation of Zuclomiphene Succinate Form APO-I

A mixture of zuclomiphene (100 mg, 0.25 mmol) and succinic acid (30.7 mg, 0.26 mmol) in ethyl acetate (6 mL) was heated at 60° C. for 2 hours. The mixture was allowed to cool to room temperature and stand to crystallize. After 5 days, the solvent was decanted off and the white crystalline solid was washed with n-heptane to afford zuclomiphene succinate Form APO-I. The PXRD and DSC thermogram of a sample prepared by this method are shown in FIG. 3 and FIG. 14, respectively.

¹H NMR (DMSO-d₆, 400 MHz): δ1.01 (t, J=7.2 Hz, 6H), 2.39 (s, 4H), 2.64 (q, J=7.1 Hz, 4H), 2.88 (t, J=5.8 Hz, 2H), 4.07 (t, J=5.43 Hz, 2H), 6.94-6.97 (m, 4H), 7.13-7.14 (m, 3H), 7.21-7.28 (m, 7H).

### Example 4

### Preparation of Zuclomiphene L-Tartrate Form APO-I

A mixture of zuclomiphene (100 mg, 0.25 mmol) and L-tartaric acid (39.0 mg, 0.26 mmol) in ethyl acetate (6 mL) was heated at 60° C. for 2 hours. The mixture was allowed to cool to room temperature and stand to crystallize overnight. The solvent was decanted off and the white crystalline solid was washed with n-heptane to afford zuclomiphene L-tartrate Form APO-I. The PXRD and DSC thermogram of a sample prepared by this method are shown in FIG. 4 and FIG. 15, respectively. The sample was determined to have an IDR of 2.37 mg min⁻¹ cm⁻².

¹H NMR (DMSO-d₆, 400 MHz): δ1.07 (t, J=7.2 Hz, 6H), 2.82 (m, 4H), 3.07 (br s, 2H), 4.09 (s, 2H), 4.13 (br s, 2H), 6.94-6.99 (m, 4H), 7.13-7.14 (m, 3H), 7.21-7.28 (m, 7H)

### Example 5

### Preparation of Zuclomiphene Tosylate Form APO-I

A mixture of zuclomiphene (100 mg, 0.25 mmol) and p-toluenesulfonic acid monohydrate (44.8 mg, 0.26 mmol) in ethyl acetate (6 mL) was heated at 60° C. for 2 hours. The mixture was allowed to cool to room temperature and stand to crystallize overnight. The solvent was decanted off and the white crystalline solid was washed with n-heptane to afford zuclomiphene tosylate Form APO-I. The PXRD diffractogram and DSC thermogram of a sample prepared by this method are shown in FIG. 5 and FIG. 16, respectively.

¹H NMR (DMSO-d₆, 400 MHz): δ1.24 (t, J=7.3 Hz, 6H), 2.28 (s, 3H), 3.22-3.27 (m, 4H), 3.53-3.55 (m, 2H), 4.33 (m, 2H), 6.94-7.04 (m, 4H), 7.10-7.15 (m, 5H), 7.22-7.32 (m, 7H), 7.46 (d, J=8.2 Hz, 2H), 9.19 (s, 1H).

### Example 6

### Preparation of Zuclomiphene L-Malate Form APO-I

A mixture of zuclomiphene (100 mg, 0.25 mmol) and L-malic acid (34.9 mg, 0.26 mmol) in ethyl acetate (6 mL) was heated at 60° C. for 2 hours. The mixture was allowed to cool to room temperature and stand to crystallize overnight. The solvent was decanted off and the white crystalline solid was washed with n-heptane. The solid was recrystallized in minimal hot ethyl acetate to afford zuclomiphene L-malate Form APO-I. The PXRD diffractogram and DSC thermogram of a sample prepared by this method are shown in FIG. 6 and FIG. 17, respectively. The sample was determined to have an IDR of 3.75 mg min⁻¹ cm⁻².

¹H NMR (DMSO-d₆, 400 MHz): δ1.10 (t, J=7.2 Hz, 6H), 2.34-2.40 (dd, J=5.4, 15.5 Hz, 1 H), 2.53-2.57 (m, 1 H), 2.88-2.89 (m, 4H), 3.14 (br s, 2H), 4.04 (dd, J=5.6, 7.7 Hz, 1H), 4.18 (t, J=4.9 Hz, 2H), 6.94-7.00 (m, 4H), 7.13-7.14 (m, 3H), 7.21-7.28 (m, 7H)

### Example 7

### Preparation of zuclomiphene maleate Form APO-I

A mixture of zuclomiphene (100 mg, 0.25 mmol) and maleic acid (30.2 mg, 0.26 mmol) in ethyl acetate (6 mL) was heated at 60° C. for 2 hours. The mixture was allowed to cool to room temperature and stand to crystallize overnight. The solvent was decanted off and the white crystalline solid was washed with n-heptane. The solid was recrystallized in minimal hot ethyl acetate to afford zuclomiphene maleate Form APO-I. The PXRD diffractogram and DSC thermogram of a sample prepared by this method are shown in FIG. 7 and FIG. 18, respectively.

¹H NMR (DMSO-d₆, 400 MHz): δ1.24 (t, J=6.4 Hz, 6H), 3.24 (br s, 4H), 3.54 (br s, 2H), 4.32 (br s, 2H), 6.01 (s, 2H), 6.94-6.97 (m, 2H), 7.03 (d, J=8.8 Hz, 2H), 7.13-7.16 (m, 3H), 7.22-7.32 (m, 7H), 9.19 (s, 1H).

### Example 8

### Preparation of Zuclomiphene Malonate Form APO-I

A mixture of zuclomiphene (100 mg, 0.25 mmol) and malonic acid (27.1 mg, 0.26 mmol) in ethyl acetate (6 mL) was heated at 60° C. for 2 hours. The mixture was allowed to cool to room temperature and stand to crystallize. After 3 days, the solvent was decanted off and the white crystalline solid was washed with n-heptane. The solid was recrystallized in minimal hot ethyl acetate to afford zuclomiphene malonate Form APO-I. The PXRD diffractogram and DSC thermogram of a sample prepared by this method are shown in FIG. 8 and FIG. 19, respectively.

¹H NMR (DMSO-d₆, 400 MHz): δ1.16 (t, J=7.2 Hz, 6H), 2.83 (s, 2H), 3.04 (br s, 4H), 3.31 (br s, 2H), 4.24 (m, 2H), 6.94-7.02 (m, 4H), 7.13-7.15 (m, 3H), 7.22-7.30 (m, 7H).

### Example 9

### Preparation of Zuclomiphene Fumarate Form APO-I

A mixture of zuclomiphene (100 mg, 0.25 mmol) and fumaric acid (30.2 mg, 0.26 mmol) in ethyl acetate (6 mL) was heated at 60° C. for 2 hours. The mixture was allowed to cool to room temperature and stand to crystallize overnight. The solvent was decanted off and the white solid was washed with n-heptane. The solid was recrystallized in minimal hot ethyl acetate to afford zuclomiphene fumarate Form APO-I. The PXRD diffractogram and DSC thermogram of a sample prepared by this method are shown in FIG. 9 and FIG. 20, respectively.

¹H NMR (DMSO-d₆, 400 MHz): δ1.02 (t, J=7.1 Hz, 6H), 2.65 (q, J=7.1 Hz, 4H), 2.89 (t, J=5.8 Hz, 2H), 4.08 (t, J=6.1 Hz, 2H), 6.58 (s, 2H), 6.94-6.98 (m, 4H), 7.13-7.15 (m, 3H), 7.21-7.26 (m, 7H).

### Example 10

### Preparation of Zuclomiphene Glycolate Form APO-I

A mixture of zuclomiphene (100 mg, 0.25 mmol) and glycolic acid (19.8 mg, 0.26 mmol) in ethyl acetate (6 mL) was heated at 60° C. for 2 hours. The mixture was allowed to cool to room temperature and stand to crystallize. After 10 days, the solvent was decanted off and the white crystalline solid was washed with n-heptane. The solid was recrystallized in minimal hot ethyl acetate to afford zuclomiphene glycolate Form APO-I. The PXRD diffractogram and DSC thermogram of a sample prepared by this method are shown in FIG. 10 and FIG. 21, respectively.

¹H NMR (DMSO-d₆, 400 MHz): δ1.00 (t, J=7.1 Hz, 6H), 2.61 (q, J=7.1 Hz, 4H), 2.84 (t, J=6.1 Hz, 2H), 3.86 (s, 2H), 4.05 (t, J=6.1 Hz, 2H), 6.94-6.97 (m, 4H), 7.12-7.15 (m, 3H), 7.21-7.28 (m, 7H).

### Example 11

### Preparation of Zuclomiphene Hemi-Citrate Form APO-I

Zuclomiphene citrate (100 mg, 0.17 mmol) and isonicotinamide (20 mg, 0.16 mmol) were dissolved in aqueous ethanol (50% v/v, 20 mL) and placed in a 20 mL scintillation vial. The vial was left open and allowed to evaporate at room temperature. After 7 days, crystals had grown at the bottom of the vial. The crystals were collected via filtration and washed on the filter with cold (0-5° C.) ethanol (5 mL). The PXRD diffractogram and DSC thermogram of a sample prepared by this method are shown in FIG. 11 and FIG. 22, respectively.

¹H NMR (DMSO-d₆, 400 MHz): δ1.10 (t, J=7.0 Hz, 6H), 2.50 (d, J=14.4 Hz, 1 H), 2.58 (d, J=15.2 Hz, 1 H), 2.87 (q, J=6.8 Hz, 4H), 3.13 (br s, 2H), 4.18 (t, J=5.0 Hz, 2H), 6.95-6.99 (m, 4H), 7.14-7.15 (m, 3H), 7.22-7.29 (m, 7H), 11.02 (vbr s, 1H)

### Example 12

### Preparation of Zuclomiphene Free Base

Zuclomiphene binaphthyl hydrogen phosphate (5 g, 6.63 mmol) and potassium carbonate (1.1 g, 7.96 mmol) were combined in a mixture of ethyl acetate and a minimum amount of water. After stirring for 5 hours at room temperature, the suspension was filtered through a pad of diatomaceous earth and the solid was washed with ethyl acetate. The combined wash and filtrate was washed with saturated potassium carbonate and brine. The organic phase was evaporated in vacuo to afford zuclomiphene free base (2.76 g, 81% yield).

¹H NMR (DMSO-d_{6, 400} MHz): δ1.07 (t, J=6.8 Hz, 6H), 2.64 (q, J=7.2, 4H), 2.89 (t, J=6.4 Hz, 2H), 4.07 (t, J=6.4 Hz, 2H), 6.88-6.91 (m, 2H), 6.95-6.97 (m, 2H), 7.07-7.08 (m, 3H), 7.09-7.10 (m, 3H), 7.15-7.28 (m, 4H).

What is claimed is:

1. A salt of zuclomiphene selected from the group consisting of zuclomiphene sulphate, zuclomiphene phosphate, zuclomiphene succinate, zuclomiphene L-tartrate, zuclomiphene tosylate, zuclomiphene L-malate, zuclomiphene maleate, zuclomiphene malonate, zuclomiphene fumarate, zuclomiphene glycolate, and zuclomiphene hemi-citrate.

2. The sulphate salt of claim 1, characterized by a PXRD diffractogram comprising peaks, expressed in degrees 2θ (±0.2°), at 5.2°, 14.6° and 20.6°.

3. The sulphate salt of claim 2, further comprising at least three peaks in the PXRD diffractogram, expressed in degrees 2θ (±0.2°), selected from the group consisting of: 10.0°, 10.3°, 12.2°, 12.4°, 12.9°, 15.1°, 16.0°, 17.4°, 22.2° and 22.9°.

4. The sulphate salt of claim 2, characterized by a DSC thermogram comprising an endothermic peak with a peak onset at approximately 152° C. and a peak maximum at approximately 155° C.

5. The phosphate salt of claim 1, characterized by a PXRD diffractogram comprising peaks, expressed in degrees 2θ (±0.2°), at 4.5°, 9.0° and 19.3°.

6. The succinate salt of claim 1, characterized by a PXRD diffractogram comprising peaks, expressed in degrees 2θ (±0.2°), at 5.5°, 10.1° and 17.3°.

7. The L-tartrate salt of claim 1, characterized by a PXRD diffractogram comprising peaks, expressed in degrees 2θ (±0.2°), at 6.0°, 9.0° and 12.0°.

8. The tosylate salt of claim 1, characterized by a PXRD diffractogram comprising peaks, expressed in degrees 2θ (±0.2°), at 5.6°, 11.1° and 18.1°.

9. The L-malate salt of claim 1, characterized by a PXRD diffractogram comprising peaks, expressed in degrees 2θ (±0.2°), at 6.4°, 12.8° and 22.5°.

10. The maleate salt of claim 1, characterized by a PXRD diffractogram comprising peaks, expressed in degrees 2θ (±0.2°), at 6.6°, 13.2° and 20.5°.

11. The malonate salt of claim 1, characterized by a PXRD diffractogram comprising peaks, expressed in degrees 2θ (±0.2°), at 6.8°, 13.6° and 18.2°.

12. The fumarate salt of claim 1, characterized by a PXRD diffractogram comprising peaks, expressed in degrees 2θ (±0.2°), at 6.9°, 13.9° and 17.9°.

13. The glycolate salt of claim 1, characterized by a PXRD diffractogram comprising peaks, expressed in degrees 2θ (±0.2°), at 6.0°, 9.0° and 18.2°.

14. The hemi-citrate salt of claim 1, characterized by a PXRD diffractogram comprising peaks, expressed in degrees 2θ (±0.2°), at 5.0°, 13.3° and 16.8°.

15. The hemi-citrate salt of claim 14, further comprising at least three peaks in the PXRD diffractogram, expressed in degrees 2θ (±0.2°), selected from the group consisting of: 9.5°, 10.9°, 14.6°, 15.7°, 18.2°, 20.2°, 20.9°, 21.6° and 24.0.

16. The hemi-citrate salt of claim 14, characterized by a DSC thermogram comprising an endothermic peak with a peak onset at approximately 82° C. and a peak maximum at approximately 86° C.

17. A pharmaceutical composition comprising a salt of zuclomiphene according to claim 1, and one or more pharmaceutically acceptable excipients.

18. The pharmaceutical composition of claim 17, wherein the pharmaceutical composition is a capsule or a tablet.

19. A method for treating a disorder selected from the group consisting of osteoporosis, bone fractures, loss of bone mineral density (BMD) and hot flashes comprising administering an effective amount of a salt of claim 1.

20. The method of claim 19, wherein the treatment comprises suppressing or inhibiting hot flashes in a male patient undergoing androgen deprivation therapy for the treatment of prostate cancer.
